(19) United States
(12) Patent Application Publication
(10) Pub. No.: US 2015/0135582 A1
(43) Pub. Date: May 21, 2015

(54) ESTER COMPOUND OF A POLYOL AND FATTY ACID OLIGOMER FOR USE AS A COLD FLOW IMPROVER IN FUEL COMPOSITIONS

(71) Applicant: DuPont Nutrition Biosciences ApS, Copenhagen K (DK)

(72) Inventors: Jens Mogens Nielsen, Galten (DK); Maja Duelund, Odder (DK); Kirsten Braüner Nygaard, Silkeborg (DK); Allan Torben Bech, Brabrand (DK); Flemming Vang Sparsø, Brabrand (DK)

(21) Appl. No.: 14/402,048

(22) PCT Filed: May 16, 2013
(86) PCT No.: PCT/EP2013/060174

§ 371 Date: Nov. 18, 2014

(30) Foreign Application Priority Data
May 18, 2012 (GB) 1208795.3

(51) Int. Cl. C10L 1/198; C10L 1/14; C07C 69/675; C10L 10/16

(52) U.S. Cl. CPC C10L 1/1986; C10L 10/16; C10L 1/143; C07C 69/675; C10L 1/146; C10L 2200/0476; C10L 2270/026; C10L 2200/0469; C10L 2300/20; C10L 2300/30; C10L 2290/24; C10L 2250/04; C10L 1/1905
USPC 44/386; 554/122

(57) ABSTRACT

The present invention provides a compound as a cold flow improver for a fuel composition, said compound being an ester of (i) a polyol wherein the polyol is selected from at least pentaerythritol, polymers thereof and mixtures thereof; and (ii) a fatty acid oligomer, wherein the fatty acid oligomer has a degree of polymerisation from 2 to 7. The ester compound can be used for reducing, preventing or inhibiting cold filter plugging in a diesel engine.

The present invention relates to a compound. In particular the present invention relates to a composition containing the compound, compositions prepared with the compound and compositions and use of the compound and compositions as cold flow improvers.

### Cold Flow Improvers

When hydrocarbon based materials such as diesel fuel and heating oils are cooled to temperatures below their cloud points, paraffinic wax crystals form within the fuel. The now widespread practice of introducing fatty acid methyl esters (FAME) into hydrocarbon based materials such as diesel fuel can lead to additional crystal formation from saturated FAME. Without the addition of appropriate cold flow additives, these wax crystals can cause a number of problems.

At temperatures close to the cloud point, filter systems such as the fuel system filters in vehicle and static installations may rapidly become blocked. At only lower temperatures, an interlocking wax crystal structure forms that prevents flow within the filter and if applicable the fuel system (i.e. the cold filter plugging point is reached)

Cold flow improvers are added to hydrocarbon based materials which are subject to such problems. Cold flow improvers act by modifying the size and/or shape of wax crystals, which in turn reduces the tendency to block filters and lines, extend the temperature range over which the hydrocarbon based materials can be used and in the case of fuels extend the temperature range over which a vehicle can operate (as measured by CFPP and other cold flow performance tests), improves operability, reduces wax settling (particularly when used with a wax anti-settling additives), and/or lowers fuel pour point and improve fuel handling.

The increased use of biofuels such as biodiesels has placed further demands on the known cold flow improvers. Furthermore, there is a desire in markets generally to replace synthetic products with those derived from natural materials. Such food based materials are often considered by consumers to be more natural than complex synthetic materials.

As discussed in US2011/0232159 surfactants are commonly used at low concentrations in commercial biodiesel additive packages to modify the size and/or shape of the crystals formed. In US2011/0232159 a total of twelve purchased/commercial surfactants and five synthesised surfactants were assessed for inclusion in polymer/biodiesel formulations by DSC and CFPP at 1% w/w concentration in biodiesel. Many of the surfactants were reported not to dissolve well in biodiesel (without warming or the use of a solvent). The surfactants tested were classified into two groups: those that dissolved and those that did not. Polyglycerol polyricinoleic (PGPR) was disclosed as one possible surfactant and was said to be soluble in biodiesel. However sucrose myristate was disclosed as being selected for further investigation in biodiesel/petro diesel blends as it was said to lower the saturated enthalpy of crystallisation to a greater degree than the other cloud point-lowering surfactants.

The present invention addresses the problems of providing a cold flow improver which is effective in hydrocarbon based materials such as diesel fuel and/or heating oils, and in particular in biodiesel, and which may be prepared from source materials typically associated with the production of food products.

In one aspect the present invention provides a compound which is an ester of

(i) a polyol wherein the polyol is selected from at least pentaerythritol, polymers thereof and mixtures thereof; and

(ii) a fatty acid oligomer, wherein the fatty acid oligomer has a degree of polymerisation of from 2 to 7.

In one aspect the present invention provides a composition comprising

(a) a compound which is an ester of
(i) a polyol wherein the polyol is selected from at least pentaerythritol, polymers thereof and mixtures thereof; and
(ii) a fatty acid oligomer, wherein the fatty acid oligomer has a degree of polymerisation of from 2 to 7

and

(b) a citric acid ester of a monoglyceride

or

(c) a copolymer of ethylene and an alkyl acrylate.

In one aspect the present invention provides a cold flow improver comprising

(A) a compound which is an ester of
(i) a polyol wherein the polyol is selected from at least pentaerythritol, polymers thereof and mixtures thereof; and
(ii) a fatty acid oligomer, wherein the fatty acid oligomer has a degree of polymerisation of from 2 to 7

or

(B) a composition comprising

(a) a compound which is an ester of
(i) a polyol wherein the polyol is selected from at least pentaerythritol, polymers thereof and mixtures thereof; and
(ii) a fatty acid oligomer, wherein the fatty acid oligomer has a degree of polymerisation of from 2 to 7

and

(b) a citric acid ester of a monoglyceride

or

(c) a copolymer of ethylene and an alkyl acrylate.

In one aspect the present invention provides a fuel composition comprising:

a fuel; and

(A) a compound which is an ester of
(I) a polyol wherein the polyol has at least three hydroxyl groups; and
(II) a fatty acid oligomer, wherein the fatty acid oligomer has a degree of polymerisation of from 2 to 7, and wherein the fatty acid oligomer is prepared from a mixture of at least
   (i) a saturated fatty acid having a hydroxyl group on the carbon chain of the fatty acid
   (ii) an unsaturated fatty acid having a hydroxyl group on the carbon chain of the fatty acid

or

(B) a composition comprising
(a) a compound which is an ester of
(I) a polyol wherein the polyol has at least three hydroxyl groups; and
(II) a fatty acid oligomer, wherein the fatty acid oligomer has a degree of polymerisation of from 2 to 7, and wherein the fatty acid oligomer is prepared from a mixture of at least
   (i) a saturated fatty acid having a hydroxyl group on the carbon chain of the fatty acid,
   (ii) an unsaturated fatty acid having a hydroxyl group on the carbon chain of the fatty acid
and
(b) a citric acid ester of a monoglyceride or
(c) a copolymer of ethylene and an alkyl acrylate.

In one aspect the present invention provides a process for reducing, preventing or inhibiting cold filter plugging in a diesel engine, comprising the step of: dosing a fuel with

(A) a compound which is an ester of

(I) a polyol wherein the polyol has at least three hydroxyl groups; and

(II) a fatty acid oligomer, wherein the fatty acid oligomer has a degree of polymerisation of from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid

or

(B) a composition comprising

(a) a compound which is an ester of

(I) a polyol wherein the polyol has at least three hydroxyl groups; and

(II) a fatty acid oligomer, wherein the fatty acid oligomer has a degree of polymerisation of from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid

and

(b) a citric acid ester of a monoglyceride

or

(c) a copolymer of ethylene and an alkyl acrylate.

In one aspect the present invention provides use of

(A) a compound which is an ester of
(I) a polyol wherein the polyol has at least three hydroxyl groups; and
(II) a fatty acid oligomer, wherein the fatty acid oligomer has a degree of polymerisation of from 2 to 7, and wherein the fatty acid oligomer is prepared from a mixture of at least
   (i) a saturated fatty acid having a hydroxyl group on the carbon chain of the fatty acid,
   (ii) an unsaturated fatty acid having a hydroxyl group on the carbon chain of the fatty acid

or

(B) a composition comprising
(a) a compound which is an ester of
   (I) a polyol wherein the polyol has at least three hydroxyl groups; and
   (II) a fatty acid oligomer, wherein the fatty acid oligomer has a degree of polymerisation of from 2 to 7, and wherein the fatty acid oligomer is prepared from a mixture of at least
      (i) a saturated fatty acid having a hydroxyl group on the carbon chain of the fatty acid,
      (ii) an unsaturated fatty acid having a hydroxyl group on the carbon chain of the fatty acid
and
(b) a citric acid ester of a monoglyceride
or
(c) a copolymer of ethylene and an alkyl acrylate
   for reducing, preventing or inhibiting cold filter plugging in a diesel engine.

For ease of reference these and further aspects of the present invention are now discussed under appropriate section headings. However, the teachings under each section are not necessarily limited to each particular section.

### Compound

The compound of the present invention is an ester of

(i) a polyol wherein the polyol is selected from at least pentaerythritol, polymers thereof and mixtures thereof; and

(ii) a fatty acid oligomer, wherein the fatty acid oligomer has a degree of polymerisation of from 2 to 7.

The compound for use in process and use of the present invention is an ester of

(I) a polyol wherein the polyol has at least three hydroxyl groups; and

(II) a fatty acid oligomer, wherein the fatty acid oligomer has a degree of polymerisation of from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid.

### Polyol

As is understood by one skilled in the art, an ester of a polyol and a fatty acid oligomer is a compound having a polyol ‘backbone’ onto which fatty acid oligomer side chains are attached.

Polyol esters of fatty acids oligomers are typically prepared by polymerisation of the polyol, for example, polymerisation of glycerol, to provide one or more polyols to which the fatty acid oligomers are then attached. The fatty acids oligomers are generally attached by direct attachment of the fatty acid oligomers to the polyol.

When the polyol is a polymer of an alcohol such as a polyglycerol, the polymerisation typically provides a mixture of polyols of different degrees of polymerisation. The mixture of polyols (e.g. polyglycerols) of different degrees of polymerisation is described herein as a polyol (e.g. polyglycerol) composition. It will be understood by one skilled in the art that references to a polyol (e.g. polyglycerol) composition having particular polyol (e.g. polyglycerol) components requires only that those components be present in the amount specified. It will be appreciated by one skilled in the art that because of the nature of polymerisation of alcohols such as glycerol, the polyol (e.g. polyglycerol) composition may contain other polyols (e.g. polyglycerols) having degrees of polymerisation not recited herein. In determining the amounts of polyols (e.g. polyglycerols) in the polyol (e.g. polyglycerol) composition, the total amount of all polyols (e.g. polyglycerols) (irrespective of degree of polymerisation) is determined to provide the total weight of the polyol (e.g. polyglycerol) composition. Materials which are not a polyol (e.g. not a polyglycerol) do not form part of the polyol (e.g. polyglycerol) composition and their weight is not considered when determining the total weight of the polyol (e.g. polyglycerol) composition.

References in the present specification to “the combined weight of the polyols (e.g. polyglycerols)” encompass the total combined weight of all polyols (e.g. polyglycerols), irrespective of their chain length and irrespective of whether the polyol (e.g. polyglycerol) is recited in the listing of polyols (e.g. polyglycerols).

In one aspect the polyol is a polyglycerol. It will be appreciated by one skilled in the art that polyglycerols may be either in the form of a cyclic polyglycerol or an acyclic polyglycerol. Acyclic polyglycerols are straight chain and branched chain polyglycerols, that is acyclic polyglycerols are formed entirely from glycerol groups linked such that no rings are formed. Cyclic polyglycerols contain a ring structure. References in the present specification to a polyglycerol of a particular degree of polymerisation, for example triglycerol referring to a polyglycerol having an average degree of polymerisation of 3, include both the polyglycerol in cyclic form and in acyclic form.

In one aspect and particular in respect of the compound of the present invention, the polyol is at least pentaerythritol, polymers thereof and mixtures thereof.

As is understood by one skilled in the art, pentaerythritol is a compound of the formula

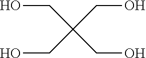

In one aspect the polyol is at least polypentaerythritol.

In one aspect the polymer of pentaerythritol has a degree of polymerisation of from greater than 1 to no greater than 10. In one aspect the polymer of pentaerythritol has a degree of polymerisation of from 2 to 10. In one aspect the polymer of pentaerythritol has a degree of polymerisation of from greater than 1 to no greater than 5. In one aspect the polymer of pentaerythritol has a degree of polymerisation of from 2 to 5. In one aspect the polymer of pentaerythritol has a degree of polymerisation of from greater than 1 to no greater than 4. In one aspect the polymer of pentaerythritol has a degree of polymerisation of from greater than 1 to no greater than 3. In one aspect the polymer of pentaerythritol has a degree of polymerisation of from greater than 1 to no greater than 2. In one aspect the polymer of pentaerythritol has a degree of polymerisation of from 1.1 to 10. In one aspect the polymer of pentaerythritol has a degree of polymerisation of from 1.1 to 5. In one aspect the polymer of pentaerythritol has a degree of polymerisation of from 1.1 to 4. In one aspect the polymer of pentaerythritol has a degree of polymerisation of from 1.1 to 3.

In one aspect the polyol is selected from at least pentaerythritol, polymers thereof and mixtures thereof; and further comprises a polyol selected from glycerol, polymers thereof and mixtures thereof.

In one aspect polyol is a mixture of at least glycerol and pentaerythritol. In one aspect polyol is a mixture of at least glycerol and dipentaerythritol. In one aspect polyol is a polymer of at least glycerol and pentaerythritol. In one aspect polyol is a polymer of at least glycerol and dipentaerythritol.

In one aspect the polyol has a hydroxyl value of from 850 to 1830, preferably from 950 to 1300.

In one aspect the polyol has a longest chain length of carbons and oxygen from 7 to 50 atoms. In one aspect the polyol has a longest chain length of carbons and oxygen from 7 to 30 atoms. In one aspect the polyol has a longest chain length of carbons and oxygen from 7 to 20 atoms. In one aspect the polyol has a longest chain length of carbons and oxygen from 7 to 15 atoms.

In one aspect the polyol has from 3 to 12 hydroxyl groups, preferably from 3 to 10 hydroxyl groups.

In one aspect the polyol comprises at least polypentaerythritol.

In one aspect the polyol comprises at least one polyol selected from dipentaerythritol, tripentaerythritol, and combinations thereof.

In one aspect the polyol is at least dipentaerythritol. As is understood by one skilled in the art, dipentaerythritol is a compound of the formula

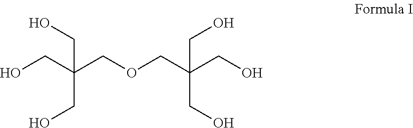 [SMILES: OCC(CO)(CO)COCC(CO)(CO)CO]

In one aspect the polyol further comprises glycerol.

In one aspect the polyol further comprises polyglycerol.

In one aspect the polyol is at least a mixture of dipentaerythritol and glycerol.

In one aspect the polyol is at least a compound of Formula I

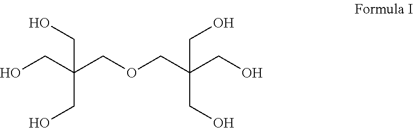 [SMILES: OCC(CO)(CO)COCC(CO)(CO)CO]

Preferably the polyol is at least a compound of Formula I in an amount of at least 50 wt % based on the amount of polyols. Other polyols may of course be present. Preferably the polyol is at least a compound of Formula I in an amount of at least 60 wt % based on the amount of polyols, such as in an amount of at least 70 wt % based on the amount of polyols, such as in an amount of at least 80 wt % based on the amount of polyols.

In the aspects of the present invention, such as in the fuel composition the ester is an ester of
(I) a polyol wherein the polyol has at least three hydroxyl groups; and
(II) a fatty acid oligomer, wherein the fatty acid oligomer has a degree of polymerisation from 2 to 7, and wherein the fatty acid oligomer is prepared from a mixture of at least
   (i) a saturated fatty acid having a hydroxyl group on the carbon chain of the fatty acid,
   (ii) an unsaturated fatty acid having a hydroxyl group on the carbon chain of the fatty acid.

Preferably the polyol is a polymer of an alcohol.

In one aspect the polymer of the alcohol has a degree of polymerisation of from greater than 1 to no greater than 10. In some aspects the polymer of the alcohol has a degree of polymerisation from 2 to 10. In one aspect the polymer of alcohol has a degree of polymerisation of from greater than 1 to no greater than 5. In one aspect the polymer of alcohol has a degree of polymerisation of from 2 to 5. In one aspect the polymer of alcohol has a degree of polymerisation of from greater than 1 to no greater than 4. In one aspect the polymer of alcohol has a degree of polymerisation of from greater than 1 to no greater than 3. In one aspect the polymer of alcohol has a degree of polymerisation of from greater than 1 to no greater than 2. In one aspect the polymer of alcohol has a degree of polymerisation of from 1.1 to 10. In one aspect the polymer of alcohol has a degree of polymerisation of from 1.1 to 5. In one aspect the polymer of alcohol has a degree of polymerisation of from 1.1 to 4. In one aspect the polymer of alcohol has a degree of polymerisation of from 1.1 to 3. In some aspects the polyol is a polymer of at least pentaerythritol. In some aspects the polyol is a polymer of at least glycerol.

In some aspects the polyol is a mixture of at least glycerol and pentaerythritol. In some aspects the polyol is a polymer of at least glycerol and pentaerythritol. In some aspects the polyol is a mixture of at least glycerol and dipentaerythritol. In some aspects the polyol is a polymer of at least glycerol and dipentaerythritol.

In some aspects the polyol is branched polyol.

In some aspects the polyol has a hydroxyl value of from 850 to 1830, preferably from 950 to 1300.

In some aspects the polyol has a longest chain length of carbons and oxygen of from 7 to 30 atoms.

In some aspects the polyol has from 3 to 12 hydroxyl groups, preferably from 3 to 10 hydroxyl groups.

### Fatty Acid Oligomer

It will be appreciated by one skilled in the art that an oligomer is a material consisting of a number of repeating units. It is distinguished from a polymer in that it has relatively few repeating units. In the present specification, and oligomer may be interpreted to mean a compound containing no greater than 30 monomer or co-monomer units.

In one aspect of the present invention the fatty acid oligomer has a degree of polymerisation of from 2 to 6.

In one aspect of the present invention the fatty acid oligomer has a degree of polymerisation from 2 to 5.

In one aspect of the present invention the fatty acid oligomer is prepared from at least one fatty acid having from 2 to 30 carbon atoms. In one aspect of the present invention the fatty acid oligomer is prepared from at least one fatty acid having from 2 to 26 carbon atoms. In one aspect of the present invention the fatty acid oligomer is prepared from at least one fatty acid having from 2 to 22 carbon atoms. In one aspect of the present invention the fatty acid oligomer is prepared from at least one fatty acid having from 6 to 22 carbon atoms.

The fatty acids of the fatty acid oligomer attached to the polyol may be of any suitable length. The polyol ester of a fatty acid oligomer may be a polyol ester of a single fatty acid oligomer, or polyol ester of an oligomer of a mixture of fatty acids. The fatty acid chain lengths of the fatty acids oligomer of the polyol ester need not be of the same length. Typically the polyol ester of the fatty acid oligomer is an ester of an oligomer of C12 to C22 fatty acid. Preferably the polyol ester of a fatty acid oligomer is an ester of an oligomer of a C16 or C18 fatty acid. Preferably the polyol ester of a fatty acid oligomer is an ester of an oligomer of a C16 and C18 fatty acid. Preferably the polyol ester of a fatty acid oligomer is an ester of an oligomer of a C18 fatty acid.

The fatty acid of the fatty acid oligomer may be saturated fatty acid, unsaturated fatty acid or a mixture of saturated fatty acid and unsaturated fatty acid. In one aspect the fatty acid of the fatty acid oligomer is an unsaturated fatty acid. The fatty acid of the fatty acid oligomer may be mono or di unsaturated fatty acid. Preferably the fatty acid of the fatty acid oligomer is a mono unsaturated fatty acid.

In one aspect of the present invention the fatty acid oligomer is prepared from at least one fatty acid having a hydroxyl group on the carbon chain of the fatty acid.

In one aspect of the present invention the fatty acid oligomer is prepared from a mixture of at least

(i) a fatty acid having a hydroxyl group on the carbon chain of the fatty acid and

(ii) an analogous fatty acid without said hydroxyl substitution.

By ‘analogous fatty acid’ it is meant a fatty acid that is of the same chain length, and if unsaturated, the same degree, position and configuration of unsaturation, as the fatty acid to which it is analogous, the sole difference being the absence of the hydroxyl substitution, the hydroxyl substitution being replaced by a hydrogen.

The fatty acids of the fatty acid oligomer may be provided from any suitable source. Thus in one aspect, the fatty acid oligomer is prepared from fatty acids from oils selected from rape seed oil, high oleic rape seed oil, soy oil, high oleic sunflower oil, tall oil fatty acids and mixtures thereof.

In a preferred aspect, the fatty acid oligomer is prepared from hydroxyl fatty acids of hydrogenated, partial hydrogenated, non-hydrogenated castor oil or mixtures thereof.

In one aspect of the present invention the fatty acid oligomer is prepared from a mixture of at least

(i) a C18-OH fatty acid (for example in an amount of approximately 85 wt % based on the total weight of C18 fatty acids used to prepare the fatty acid oligomer) having a hydroxyl group on the carbon chain of the fatty acid and

(ii) a C18 fatty acid (for example in an amount of approximately 15 wt % based on the total weight of C18 fatty acids used to prepare the fatty acid oligomer) without said hydroxyl substitution.

In one aspect of the present invention the fatty acid oligomer is prepared from at least an unsaturated fatty acid having a hydroxyl group on the carbon chain of the fatty acid.

In one aspect of the present invention the fatty acid oligomer is prepared from at least an unsaturated fatty acid having a hydroxyl group on the carbon chain of the fatty acid, wherein the unsaturated fatty acid having a hydroxyl group on the carbon chain of the fatty acid is present in an amount of no greater than 50 wt %, such as in an amount of no greater than 45 wt %, such as in an amount of no greater than 40 wt %, such as in an amount of no greater than 35 wt %, such as in an amount of no greater than 30 wt %, such as in an amount of no greater than 25 wt %, such as in an amount of no greater than 20 wt %, such as in an amount of no greater than 15 wt %, such as in an amount of no greater than 10 wt %, such as in an amount of no greater than 5 wt %, based on the total weight of fatty acids used to prepare the fatty acid oligomer.

In one aspect of the present invention the fatty acid oligomer is prepared from a mixture of at least

(i) a saturated fatty acid having a hydroxyl group on the carbon chain of the fatty acid, (for example in an amount of approximately 80 wt % based on the total weight of fatty acids used to prepare the fatty acid oligomer)

(ii) an unsaturated fatty acid having a hydroxyl group on the carbon chain of the fatty acid. (for example in an amount of approximately 20 wt % based on the total weight of fatty acids used to prepare the fatty acid oligomer)

In one aspect of the present invention the fatty acid oligomer is prepared from at least 12-hydroxy stearic acid.

In one aspect of the present invention the fatty acid oligomer is prepared from at least ricinoleic acid.

In one aspect of the present invention the fatty acid oligomer is prepared from a mixture of at least

(i) 12-hydroxy stearic acid and

(ii) ricinoleic acid.

In one aspect of the present invention the fatty acid oligomer is prepared from a mixture of at least

(i) 12-hydroxy stearic acid in an amount of 60-90 wt % based on the total weight of fatty acids used to prepare the fatty acid oligomer and

(ii) ricinoleic acid in an amount of 10-40 wt % based on the total weight of fatty acids used to prepare the fatty acid oligomer.

In one aspect of the present invention the fatty acid oligomer is prepared from a mixture of at least

(i) 12-hydroxy stearic acid in an amount of 70-90 wt % based on the total weight of fatty acids used to prepare the fatty acid oligomer and

(ii) ricinoleic acid in an amount of 10-30 wt % based on the total weight of fatty acids used to prepare the fatty acid oligomer.

In one aspect of the present invention the fatty acid oligomer is prepared from a mixture of at least

(i) 12-hydroxy stearic acid in an amount of 75-85 wt % based on the total weight of fatty acids used to prepare the fatty acid oligomer and

(ii) ricinoleic acid in an amount of 15-25 wt % based on the total weight of fatty acids used to prepare the fatty acid oligomer.

In one aspect of the present invention the fatty acid oligomer is prepared from a mixture of at least

(i) 12-hydroxy stearic acid in an amount of approximately 80 wt % based on the total weight of fatty acids used to prepare the fatty acid oligomer and

(ii) ricinoleic acid in an amount of approximately 20 wt % based on the total weight of fatty acids used to prepare the fatty acid oligomer.

In one aspect of the present invention the fatty acid oligomer is prepared from a mixture which further comprises a fatty acid group which does not contain a hydroxyl group on the fatty acid chain.

In one aspect of the present invention the fatty acid oligomer is prepared from a mixture of at least

(i) an unsaturated fatty acid having a hydroxyl group on the carbon chain of the fatty acid (for example in an amount of approximately 85 wt % based on the total weight of fatty acids used to prepare the fatty acid oligomer) and

(ii) an analogous unsaturated fatty acid without said hydroxyl substitution (for example in an amount of approximately 15 wt % based on the total weight of fatty acids used to prepare the fatty acid oligomer).

In one aspect of the present invention the fatty acid oligomer is prepared from a mixture of at least

(i) a saturated fatty acid having a hydroxyl group on the carbon chain of the fatty acid (for example in an amount of approximately 85 wt % based on the total weight of fatty acids used to prepare the fatty acid oligomer) and

(ii) an analogous saturated fatty acid without said hydroxyl substitution (for example in an amount of approximately 15 wt % based on the total weight of fatty acids used to prepare the fatty acid oligomer).

In one aspect of the present invention the fatty acid oligomer is prepared from a mixture of at least

(i) an unsaturated fatty acid having a hydroxyl group on the carbon chain of the fatty acid (for example in an amount of approximately 85 wt % based on the total weight of unsaturated fatty acids used to prepare the fatty acid oligomer);

(ii) an unsaturated fatty acid analogous to (i) without said hydroxyl substitution (for example in an amount of approximately 15 wt % based on the total weight of unsaturated fatty acids used to prepare the fatty acid oligomer);

(iii) a saturated fatty acid having a hydroxyl group on the carbon chain of the fatty acid (for example in an amount of approximately 85 wt % based on the total weight of saturated fatty acids used to prepare the fatty acid oligomer); and

(iv) a saturated fatty acid analogous to (iii) without said hydroxyl substitution (for example in an amount of approximately 15 wt % based on the total weight of saturated fatty acids used to prepare the fatty acid oligomer).

In one aspect of the present invention the fatty acid oligomer has a degree of polymerisation of from 2 to 5 when measured by NMR.

In one aspect of the present invention the fatty acid oligomer has an acid value of from 20 to 100, such as from 30 to 80, such as from 30 to 70, such as from 40 to 70.

In one aspect of the present invention the ratio of polyol to fatty acid oligomer based on weight is from 1:50 to 1:1. In one aspect of the present invention the ratio of polyol to fatty acid oligomer based on weight is from 1:50 to 1:4. In one aspect of the present invention the ratio of polyol to fatty acid oligomer based on weight is from 1:25 to 1:4. In one aspect of the present invention the ratio of polyol to fatty acid oligomer based on weight is from 1:50 to 1:10. In one aspect of the present invention the ratio of polyol to fatty acid oligomer based on weight is from 1:40 to 1:10. In one aspect of the present invention the ratio of polyol to fatty acid oligomer based on weight is from 1:30 to 1:10. In one aspect of the present invention the ratio of polyol to fatty acid oligomer based on weight is from 1:25 to 1:10. In one aspect of the present invention the ratio of polyol to fatty acid oligomer based on weight is from 1:25 to 1:15. In one aspect of the present invention the ratio of polyol to fatty acid oligomer based on weight is from 1:23 to 1:19.

In one aspect of the present invention the polyol is present in an amount of from 60 to 99 wt. % and the fatty acid oligomer is present in an amount of from 1 to 40 wt. %, wherein the amounts are based on the total amount of polyol and fatty acid oligomer. In one aspect of the present invention the polyol is present in an amount of from 70 to 99 wt. % and the fatty acid oligomer is present in an amount of from 1 to 30 wt. %, wherein the amounts are based on the total amount of polyol and fatty acid oligomer. In one aspect of the present invention the polyol is present in an amount of from 80 to 99 wt. % and the fatty acid oligomer is present in an amount of from 1 to 20 wt. %, wherein the amounts are based on the total amount of polyol and fatty acid oligomer. In one aspect of the present invention the polyol is present in an amount of from 90 to 99 wt. % and the fatty acid oligomer is present in an amount of from 1 to 10 wt. %, wherein the amounts are based on the total amount of polyol and fatty acid oligomer. In one aspect of the present invention the polyol is present in an amount of from 91 to 97 wt. % and the fatty acid oligomer is present in an amount of from 3 to 9 wt. %, wherein the amounts are based on the total amount of polyol and fatty acid oligomer. In one aspect of the present invention the polyol is present in an amount of approximately 96 wt. % and the fatty acid oligomer is present in an amount of approximately 4 wt. %, wherein the amounts are based on the total amount of polyol and fatty acid oligomer. In one aspect of the present invention the polyol is present in an amount of approximately 95.6 wt. % and the fatty acid oligomer is present in an amount of approximately 4.4 wt. %, wherein the amounts are based on the total amount of polyol and fatty acid oligomer.

Preferably the polyol is at least dipentaerythritol. and the fatty acid oligomer is prepared from a mixture of at least (i) 12-hydroxy stearic acid and (ii) ricinoleic acid. In this aspect, preferably the ratio of dipentaerythritol to fatty acid oligomer based on weight is from 1:50 to 1:1, in particular from 1:50 to 1:4, in particular from 1:25 to 1:4, in particular from 1:50 to 1:10, in particular from 1:40 to 1:10, in particular from 1:30 to 1:10, in particular from 1:25 to 1:10, in particular from 1:25 to 1:15, in particular from 1:23 to 1:19.

Preferably the polyol is at least dipentaerythritol and the fatty acid oligomer is prepared from a mixture of at least (i) 12-hydroxy stearic acid in an amount of 60-90 wt % based on the total weight of fatty acids used to prepare the fatty acid oligomer and (ii) ricinoleic acid in an amount of 10-40 wt % based on the total weight of fatty acids used to prepare the fatty acid oligomer. In this aspect, preferably the ratio of dipentaerythritol to fatty acid oligomer based on weight is from 1:50 to 1:1, in particular from 1:50 to 1:4, in particular from 1:25 to 1:4, in particular from 1:50 to 1:10, in particular from 1:40 to 1:10, in particular from 1:30 to 1:10, in particular from 1:25 to 1:10, in particular from 1:25 to 1:15, in particular from 1:23 to 1:19.

Preferably the polyol is at least dipentaerythritol and the fatty acid oligomer is prepared from a mixture of at least (i) 12-hydroxy stearic acid in an amount of 70-90 wt % based on the total weight of fatty acids used to prepare the fatty acid oligomer and (ii) ricinoleic acid in an amount of 10-30 wt % based on the total weight of fatty acids used to prepare the fatty acid oligomer. In this aspect, preferably the ratio of dipentaerythritol to fatty acid oligomer based on weight is from 1:50 to 1:1, in particular from 1:50 to 1:4, in particular from 1:25 to 1:4, in particular from 1:50 to 1:10, in particular from 1:40 to 1:10, in particular from 1:30 to 1:10, in particular from 1:25 to 1:10, in particular from 1:25 to 1:15, in particular from 1:23 to 1:19.

Preferably the polyol is at least dipentaerythritol and the fatty acid oligomer is prepared from a mixture of at least (i) 12-hydroxy stearic acid in an amount of 75-85 wt % based on the total weight of fatty acids used to prepare the fatty acid oligomer and (ii) ricinoleic acid in an amount of 15-25 wt % based on the total weight of fatty acids used to prepare the fatty acid oligomer. In this aspect, preferably the ratio of dipentaerythritol to fatty acid oligomer based on weight is from 1:50 to 1:1, in particular from 1:50 to 1:4, in particular from 1:25 to 1:4, in particular from 1:50 to 1:10, in particular from 1:40 to 1:10, in particular from 1:30 to 1:10, in particular from 1:25 to 1:10, in particular from 1:25 to 1:15, in particular from 1:23 to 1:19.

Preferably the polyol is at least dipentaerythritol and the fatty acid oligomer is prepared from a mixture of at least (i) 12-hydroxy stearic acid in an amount of approximately 80 wt % based on the total weight of fatty acids used to prepare the fatty acid oligomer and (ii) ricinoleic acid in an amount of approximately 20 wt % based on the total weight of fatty acids used to prepare the fatty acid oligomer. In this aspect, preferably the ratio of dipentaerythritol to fatty acid oligomer based on weight is from 1:50 to 1:1, in particular from 1:50 to 1:4, in particular from 1:25 to 1:4, in particular from 1:50 to 1:10, in particular from 1:40 to 1:10, in particular from 1:30 to 1:10, in particular from 1:25 to 1:10, in particular from 1:25 to 1:15, in particular from 1:23 to 1:19.

Preferably the polyol is dipentaerythritol present in an amount of approximately 4.4 wt. %, (based on the total amount of polyol and fatty acid oligomer) and the fatty acid oligomer is present in an amount of approximately 95.6 wt. % (based on the total amount of polyol and fatty acid oligomer) wherein the fatty acid oligomer prepared from a mixture of at least (i) 12-hydroxy stearic acid in an amount of approximately 80 wt % based on the total weight of fatty acids used to prepare the fatty acid oligomer and (ii) ricinoleic acid in an amount of approximately 20 wt % based on the total weight of fatty acids used to prepare the fatty acid oligomer.

### Compound

In one aspect of the present invention the compound is of Formula II

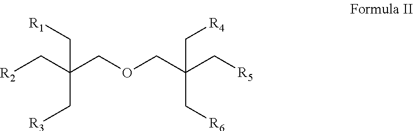

wherein each of R₁ to R₆ is independently selected from OH and fatty acid oligomer esters, wherein at least one of R₁ to R₆ is a fatty acid oligomer ester.

Preferably the compound is at least a compound of Formula II in an amount of at least 60 wt % based on the amount of esters. Other esters may of course be present. Preferably the ester is at least a compound of Formula II in an amount of at least 70 wt % based on the amount of esters, such as in an amount of at least 80 wt % based on the amount of esters.

In one aspect wherein each of R₁ to R₆ is independently selected from OH and fatty acid oligomers of Formula III

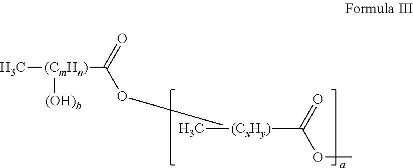

wherein b is 0 or 1, m is an integer from 0 to 28, n is selected from 2m-b, 2m-2-b, 2m-4-b, x is an integer from 0 to 28, y is selected from 2x-1, 2x-3, 2x-5, and a is an integer from 1 to 9.

In one aspect b is 0. In one aspect b is 1.

In one aspect m is an integer from 0 to 20. In one aspect m is an integer from 10 to 20. In one aspect m is an integer from 12 to 18. In one aspect m is an integer from 14 to 18. In one aspect m is 14 or 16

In one aspect n is 2m-b. In one aspect n is 2m-2-b. In one aspect n is 2m-4-b.

In one aspect x is an integer from 0 to 20. In one aspect x is an integer from 10 to 20. In one aspect x is an integer from 12 to 18. In one aspect x is an integer from 14 to 18. In one aspect x is 14 or 16

In one aspect y is 2x-1. In one aspect y is 2x-3. In one aspect y is 2x-5.

In one aspect a is from 1 to 7. In one aspect a is from 1 to 5. In one aspect a is from 1 to 4. In one aspect a is 1. In one aspect a is 2. In one aspect a is 3. In one aspect a is 4. In one aspect a is 5. In one aspect a is 6. In one aspect a is 7. In one aspect a is 8. In one aspect a is 9.

### Composition

As discussed herein, in one aspect the present invention provides a composition comprising

(a) a compound which is an ester of
(i) a polyol wherein the polyol has at least three hydroxyl groups; and
(ii) a fatty acid oligomer, wherein the fatty acid oligomer has a degree of polymerisation of from 2 to 7.

and

(b) a citric acid ester of a monoglyceride

or

(c) a copolymer of ethylene and an alkyl acrylate.

### Citrem (Citric Acid Ester of a Monoglyceride)

In one aspect the composition comprises

(a) a compound as defined herein (such as in any one of claims 1 to 13) and

(b) a citric acid ester of a monoglyceride.

As understood by one skilled in the art, no monoglyceride is a single pure substance. It typically contains a mixture of fatty acid groups attached to the glycerol backbone. Furthermore, it typically contains a mixture of mono and di glycerides. References herein to citric acid ester of a monoglyceride therefore encompass citric acid esters of monoglycerides and diglycerides.

In one aspect the citric acid ester of a monoglyceride is a citric acid ester of a monoglyceride derived from an oil selected from sunflower oil, high oleic sunflower oil and rapeseed oil.

In one aspect the ratio of (a) to (b) based on weight is from 20:1 to 1:10.

In one aspect the ratio of (a) to (b) based on weight is from 10:1 to 1:3

The fatty acids of the citric acid ester monoglyceride may be provided from any suitable source. Thus in one aspect, the citric acid ester monoglyceride is prepared from fatty acids from oils selected from rape seed oil, high oleic rape seed oil, soy oil, high oleic sunflower oil, tall oil fatty acids and mixtures thereof.

In one preferred aspect the citric acid ester of monoglyceride is provided in the composition in the form of a blend of a triglyceride and a citric acid ester of monoglyceride. The triglyceride may be provided from any suitable source. Preferred oils that may provide the source of the triglyceride are the group consisting of soy oil, rapeseed oil, soy oil, olive oil, palm olein, other vegetable oils such as Jathropha oil, and mixtures thereof The citric acid ester of monoglyceride may be blended with the triglyceride in any suitable amount and the desired amount of triglyceride may vary between the different oils. In one aspect the triglyceride is present in an amount of 5-50 wt. %, such as 5-40 wt. %, such as 5-30 wt. %, such as 10-30 wt. %, such as 15-25 wt. %, such as approximately 20 wt. %, based on the total weight of the triglyceride and the citric acid ester of monoglyceride.

### Copolymer of Ethylene and an Alkyl Acrylate

In one aspect the composition comprises

(a) a compound as defined herein (such as in any one of claims 1 to 13) and

(c) a copolymer of ethylene and an alkyl acrylate.

In one aspect the alkyl acrylate has up to 10 carbon atoms in the alkyl chain.

In one aspect the alkyl group of the alkyl acrylate is selected from methyl, ethyl, n-butyl and 2-ethylhexyl.

In one aspect the alkyl acrylate is selected from the group consisting of methyl acrylate, ethyl acrylate, n-butyl acrylate, 2-ethylhexyl acrylate and mixtures thereof.

In one aspect the alkyl acrylate is methyl acrylate.

In one aspect the copolymer is derived from copolymerization of ethylene with from 45 to 75 weight % of an alkyl acrylate wherein the copolymer has a number average molecular weight (Mn) above about 40,000 and a melt index of from 2 to 14 g/10 min.

In one aspect the copolymer further comprising a curing agent, one or more additives, or combinations thereof wherein the additive includes an antioxidant, an internal release agent, a scorch retarder, a plasticizer, an accelerator, or a filler and the composition is optionally a cured or post-cured composition.

In one aspect the copolymer further comprises at least one additional polymer, a curing agent, an additive, or combinations of two or more thereof wherein the additional polymer includes an ethylene alkyl acrylate copolymer, a polyacrylate copolymer, or combinations thereof. The additive may include an antioxidant, an internal release agent, a scorch retarder, a plasticizer, an accelerator, or a filler and optionally the composition is a cured or post-cured composition.

In one aspect the copolymer further comprises a curing agent, a second polymer, and optionally an additive and optionally the composition is a cured composition wherein the second polymer includes a thermoset, thermoplastic, or combinations thereof. The thermoset may include unsaturated polyester resin, vinyl ester resin, or combinations thereof and the additive includes filler, reinforcing fiber, fibrous structure, or combinations of two or more thereof.

In one aspect the copolymer comprises methyl acrylate and Mn from about 40,000 to about 65,000, has a melt index from 2 to 12 g/10 min, and has a polydispersity from about 3 to about 7.

In one aspect the copolymer has a polydispersity from 4 to 6.

In one aspect the copolymer is an ethylene methyl acrylate copolymer, has an Mn from about 40,000 to about 65,000, has a melt index from 2 to 12 g/10 min, and has a polydispersity from about 3 to about 7.

In one aspect the copolymer is a copolymer as described in U.S. Pat. No. 7,544,757 (incorporated herein by reference).

In one aspect the ratio of (a) to (c) based on weight is from 100:1 to 1:2.

In one aspect the ratio of (a) to (c) based on weight is from 50:1 to 1:1.

### Three Part Composition

In one aspect the composition comprises

(a) a compound as defined herein (such as in any one of claims 1 to 13);

(b) a citric acid ester of a monoglyceride; and

(c) a copolymer of ethylene and an alkyl acrylate.

In one aspect the ratio of (a) to (b) based on weight is from 20:1 to 1:10; and the ratio of (a) to (c) based on weight is from 100:1 to 1:2.

### In One Aspect

the ratio of (a) to (b) based on weight is from 10:1 to 1:3; and

the ratio of (a) to (c) based on weight is from 50:1 to 1:1.

### Fuel

The fuel may be any fuel in which cold filter plugging or wax deposition is a problem. Preferably the fuel is a fuel for a high compression spontaneous ignition engine. In one aspect the fuel is selected from diesel, heavy fuel oil, marine gasoil (MGO) and kerosene. The diesel may be biodiesel, low sulphur diesel and ultra-low sulphur diesel. Preferably the fuel is biodiesel or a biodiesel blend.

The biodiesel in one aspect is selected from the group consisting of tallow oil biodiesel soy bean oil biodiesel, rapeseed oil biodiesel, palm oil biodiesel, and mixtures thereof.

The biodiesel in one aspect is a blend of petro diesel and a biodiesel selected from the group consisting of tallow oil biodiesel, soy bean oil biodiesel, rapeseed oil biodiesel, palm oil biodiesel, and mixtures thereof.

The biodiesel may be blended with the petro diesel in any suitable amount to provide a bio/petro diesel blend. For example the biodiesel may comprise at least 1 wt % of the bio/petro diesel blend, such as at least 2 wt % of the blend, such as at least 5 wt % of the blend, such as at least 7 wt % of the blend, such as at least 10 wt % of the blend, such as at least 20 wt % of the blend, such as at least 30 wt % of the blend, such as at least 40 wt % of the blend, such as at least 50 wt % of the blend, such as at least 60 wt % of the blend, such as at least 70 wt % of the blend, such as at least 80 wt % of the blend, such as at least 90 wt % of the blend, such as at least 95 wt. % of the blend, based on the total amount of biodiesel and petro diesel.

Further the biodiesel may comprises no greater than 95 wt % of the bio/petro diesel blend, such as no greater than 90 wt % of the blend, such as no greater than 80 wt % of the blend, such as no greater than 70 wt % of the blend, such as no greater than 60 wt % of the blend, such as no greater than 50 wt % of the blend, such as no greater than 40 wt % of the blend, such as no greater than 30 wt % of the blend, such as no greater than 20 wt % of the blend, such as no greater than 10 wt % of the blend, such as no greater than 7 wt % of the blend, such as no greater than 5 wt % of the blend, such as no greater than 2 wt % of the blend, such as no greater than 1 wt % of the blend, based on the total amount of biodiesel and petro diesel.

In one aspect the diesel is solely a biodiesel selected from the group consisting of tallow oil biodiesel, soy bean oil biodiesel, rapeseed oil biodiesel, palm oil biodiesel, and mixtures thereof.

In one aspect, the ester of a polyol and a fatty acid oligomer is typically dosed into a fuel in an amount of no greater than 1 wt % of the ester, such as no greater than 0.9 wt % of the ester, such as no greater than 0.8 wt % of the ester, such as no greater than 0.7 wt % of the ester, such as no greater than 0.6 wt % of the ester, such as no greater than 0.5 wt % of the ester, such as no greater than 0.4 wt % of the ester, such as no greater than 0.3 wt. % based on the total amount of fuel.

The ester of a polyol and a fatty acid oligomer is typically dosed into a fuel in an amount of at least 0.01 wt % of the ester, such as at least 0.02 wt % of the ester, such as at least 0.03 wt % of the ester, such as at least 0.04 wt % of the ester, such as at least 0.05 wt % of the ester, such as at least 0.06 wt % of the ester, such as at least 0.07 wt % of the ester, such as at least 0.08 wt % of the ester, such as at least 0.09 wt % of the ester, such as at least 0.1 wt % of the ester, such as at least 0.12 wt % of the ester, such as at least 0.15 wt % of the ester, such as at least 0.17 wt % of the ester, such as at least 0.2 wt % of the ester, such as at least 0.25 wt % of the ester, such as at least 0.3 wt % of the ester, based on the total amount of fuel.

In one aspect, the amount of ester of a polyol and a fatty acid oligomer dosed into a fuel may be reduced based on the proportion of biodiesel present in a blend of biodiesel and petro diesel. Therefore in one aspect the fatty acid oligomer is dosed into a blend of biodiesel and petro diesel in an amount of at least 0.01 wt % of the ester, such as at least 0.02 wt % of the ester, such as at least 0.03 wt % of the ester, such as at least 0.04 wt % of the ester, such as at least 0.05 wt % of the ester, such as at least 0.06 wt % of the ester, such as at least 0.07 wt % of the ester, such as at least 0.08 wt % of the ester, such as at least 0.09 wt % of the ester, such as at least 0.1 wt % of the ester, such as at least 0.12 wt % of the ester, such as at least 0.15 wt % of the ester, such as at least 0.17 wt % of the ester, such as at least 0.2 wt % of the ester, such as at least 0.25 wt % of the ester, such as at least 0.3 wt % of the ester, based on the amount of fuel biodiesel. For example in a blend of biodiesel and petro diesel comprising 10 wt % biodiesel and 90 wt % petro diesel, the recited amounts may be divided by 10 to provide the dosage of ester based on the total amount of fuel.

In one aspect, the ester of a polyol and a fatty acid oligomer is typically dosed into a blend of biodiesel and petro diesel in an amount of no greater than 1 wt % of the ester, such as no greater than 0.9 wt % of the ester, such as no greater than 0.8 wt % of the ester, such as no greater than 0.7 wt % of the ester, such as no greater than 0.6 wt % of the ester, such as no greater than 0.5 wt % of the ester, such as no greater than 0.4 wt % of the ester, such as no greater than 0.3 wt. % based on the total amount of biodiesel.

In one aspect, the citric acid ester of a monoglyceride is typically dosed into a fuel in an amount of no greater than 1 wt % of the ester, such as no greater than 0.9 wt % of the ester, such as no greater than 0.8 wt % of the ester, such as no greater than 0.7 wt % of the ester, such as no greater than 0.6 wt % of the ester, such as no greater than 0.5 wt % of the ester, such as no greater than 0.4 wt % of the ester, such as no greater than 0.3 wt. %, such as no greater than 0.2 wt. % based on the total amount of fuel.

In one aspect, the citric acid ester of a monoglyceride is typically dosed into a fuel in an amount of at least 0.01 wt % of the ester, such as at least 0.02 wt % of the ester, such as at least 0.03 wt % of the ester, such as at least 0.04 wt % of the ester, such as at least 0.05 wt % of the ester, such as at least 0.06 wt % of the ester, such as at least 0.07 wt % of the ester, such as at least 0.08 wt % of the ester, such as at least 0.09 wt % of the ester, such as at least 0.1 wt % of the ester, such as at least 0.12 wt % of the ester, such as at least 0.15 wt % of the ester, such as at least 0.17 wt % of the ester, such as at least 0.2 wt % of the ester, based on the amount of fuel.

In one aspect, the amount of citric acid ester of a monoglyceride dosed into a fuel may be reduced based on the proportion of biodiesel present in a blend of biodiesel and petro diesel. Therefore in one aspect the citric acid ester of a monoglyceride is dosed into a fuel blend of biodiesel and petro diesel in an amount of at least 0.01 wt % of the ester, such as at least 0.02 wt % of the ester, such as at least 0.03 wt % of the ester, such as at least 0.04 wt % of the ester, such as at least 0.05 wt % of the ester, such as at least 0.06 wt % of the ester, such as at least 0.07 wt % of the ester, such as at least 0.08 wt % of the ester, such as at least 0.09 wt % of the ester, such as at least 0.1 wt % of the ester, such as at least 0.12 wt % of the ester, such as at least 0.15 wt % of the ester, such as at least 0.17 wt % of the ester, such as at least 0.2 wt % of the ester, based on the total amount of biodiesel.

In one aspect, the citric acid ester of a monoglyceride is typically dosed into a blend of biodiesel and petro diesel in an amount of no greater than 1 wt % of the ester, such as no greater than 0.9 wt % of the ester, such as no greater than 0.8 wt % of the ester, such as no greater than 0.7 wt % of the ester, such as no greater than 0.6 wt % of the ester, such as no greater than 0.5 wt % of the ester, such as no greater than 0.4 wt % of the ester, such as no greater than 0.3 wt. %, such as no greater than 0.2 wt. % based on the total amount of biodiesel.

In one aspect, the copolymer of ethylene and an alkyl acrylate is typically dosed into a fuel in an amount of no greater than 0.1 wt % of the ester, such as no greater than 0.09 wt % of the ester, such as no greater than 0.08 wt % of the ester, such as no greater than 0.07 wt % of the ester, such as no greater than 0.06 wt % of the ester, such as no greater than 0.05 wt % of the ester, such as no greater than 0.04 wt % of the ester, such as no greater than 0.03 wt. %, such as no greater than 0.02 wt. % based on the total amount of fuel.

In one aspect, the copolymer of ethylene and an alkyl acrylate is typically dosed into a fuel in an amount of at least 0.001 wt % of the ester, such as at least 0.002 wt % of the ester, such as at least 0.003 wt % of the ester, such as at least 0.004 wt % of the ester, such as at least 0.005 wt % of the ester, such as at least 0.006 wt % of the ester, such as at least 0.007 wt % of the ester, such as at least 0.008 wt % of the ester, such as at least 0.009 wt % of the ester, such as at least 0.01 wt % of the ester, such as at least 0.012 wt % of the ester, such as at least 0.015 wt % of the ester, such as at least 0.017 wt % of the ester, such as at least 0.02 wt % of the ester, based on the total amount of fuel.

In one aspect, the amount of copolymer of ethylene and an alkyl acrylate dosed into a fuel may be reduced based on the proportion of biodiesel present in a blend of biodiesel and petro diesel. Therefore in one aspect the copolymer of ethylene and an alkyl acrylate is dosed into a fuel blend of biodiesel and petro diesel in an amount of at least 0.001 wt % of the ester, such as at least 0.002 wt % of the ester, such as at least 0.003 wt % of the ester, such as at least 0.004 wt % of the ester, such as at least 0.005 wt % of the ester, such as at least 0.006 wt % of the ester, such as at least 0.007 wt % of the ester, such as at least 0.008 wt % of the ester, such as at least 0.009 wt % of the ester, such as at least 0.01 wt % of the ester, such as at least 0.012 wt % of the ester, such as at least 0.015 wt % of the ester, such as at least 0.017 wt % of the ester, such as at least 0.02 wt % of the ester, based on the total amount of biodiesel.

In one aspect, the copolymer of ethylene and an alkyl acrylate is typically dosed into a blend of biodiesel and petro diesel in an amount of no greater than 0.1 wt % of the ester, such as no greater than 0.09 wt % of the ester, such as no greater than 0.08 wt % of the ester, such as no greater than 0.07 wt % of the ester, such as no greater than 0.06 wt % of the ester, such as no greater than 0.05 wt % of the ester, such as no greater than 0.04 wt % of the ester, such as no greater than 0.03 wt. %, such as no greater than 0.02 wt. % based on the total amount of biodiesel.

The composition or fuel composition according to the present invention may comprise one or more additives for example, to improve various aspects of the fuel to which the composition is typically added or to improve various aspects of the combustion system performance. Suitable additional additives include detergents, carrier oils, anti-oxidants, corrosion inhibitors, colour stabilisers, metal deactivators, cetane number improvers, other combustion improvers, antifoams, pour point depressants, further cold filter plugging depressants, wax anti-settling additives, dispersants, reodorants, dyes, smoke suppressants, lubricity agents, and other particulate filter regeneration additives.

### Further Aspects

It will be understood by one skilled in the art that fuels are typical hydrocarbon based materials which suffer from the problems of cold flow and to which the addition of a cold flow improver is desirable. However the problem of cold flow may be exhibited in other hydrocarbon based materials. Therefore in a further aspect the present invention provides the following.

In one aspect the present invention provides a hydrocarbon composition comprising: a hydrocarbon fluid; and

(A) a compound which is an ester of
(I) a polyol wherein the polyol has at least three hydroxyl groups; and
(II) a fatty acid oligomer, wherein the fatty acid oligomer has a degree of polymerisation of from 2 to 7, and wherein the fatty acid oligomer is prepared from a mixture of at least
   (i) a saturated fatty acid having a hydroxyl group on the carbon chain of the fatty acid
   (ii) an unsaturated fatty acid having a hydroxyl group on the carbon chain of the fatty acid

or

(B) a composition comprising
(a) a compound which is an ester of
(I) a polyol wherein the polyol has at least three hydroxyl groups; and
(II) a fatty acid oligomer, wherein the fatty acid oligomer has a degree of polymerisation of from 2 to 7, and wherein the fatty acid oligomer is prepared from a mixture of at least
   (i) a saturated fatty acid having a hydroxyl group on the carbon chain of the fatty acid,
   (ii) an unsaturated fatty acid having a hydroxyl group on the carbon chain of the fatty acid
and
(b) a citric acid ester of a monoglyceride or
(c) a copolymer of ethylene and an alkyl acrylate.

In one aspect the present invention provides a process for reducing, preventing or inhibiting cold filter plugging by a hydrocarbon fluid, comprising the step of: dosing a hydrocarbon fluid with

(A) a compound which is an ester of

(I) a polyol wherein the polyol has at least three hydroxyl groups; and

(II) a fatty acid oligomer, wherein the fatty acid oligomer has a degree of polymerisation of from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid

or

(B) a composition comprising

(a) a compound which is an ester of

(I) a polyol wherein the polyol has at least three hydroxyl groups; and

(II) a fatty acid oligomer, wherein the fatty acid oligomer has a degree of polymerisation of from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid
and
(b) a citric acid ester of a monoglyceride
or
(c) a copolymer of ethylene and an alkyl acrylate.

We have also surprisingly found that in some aspects of the present invention it is not essential for the ester of a polyol and fatty acid oligomer to be present. Thus the present invention may provide:
a fuel composition,
a process for reducing, preventing or inhibiting cold filter plugging in a diesel engine, and
for reducing, preventing or inhibiting cold filter plugging in a diesel engine.

using a compound selected from a citric acid ester of a monoglyceride, a copolymer of ethylene and an alkyl acrylate and mixtures thereof.

In one further aspect the present invention provides a fuel composition comprising:

(I) a fuel; and

(II) a citric acid ester of a monoglyceride.

Preferably the fuel composition further comprises a copolymer of ethylene and an alkyl acrylate.

In one further aspect the present invention provides a hydrocarbon fluid composition comprising:

(I) a hydrocarbon fluid; and

(II) a citric acid ester of a monoglyceride.

Preferably the hydrocarbon fluid composition further comprises a copolymer of ethylene and an alkyl acrylate.

In one further aspect the present invention provides a fuel composition comprising:

(I) a fuel; and

(II) a copolymer of ethylene and an alkyl acrylate.

Preferably the fuel composition further comprises a citric acid ester of a monoglyceride.

In one further aspect the present invention provides a hydrocarbon fluid composition comprising:

(I) a hydrocarbon fluid; and

(II) a copolymer of ethylene and an alkyl acrylate.

Preferably the fuel composition further comprises a citric acid ester of a monoglyceride.

In one aspect the present invention provides a process for reducing, preventing or inhibiting cold filter plugging in a diesel engine, comprising the step of: dosing a fuel with a citric acid ester of a monoglyceride. Preferably the process further comprises the step of dosing the fuel with a copolymer of ethylene and an alkyl acrylate.

In one aspect the present invention provides a process for reducing, preventing or inhibiting cold filter plugging in a diesel engine, comprising the step of: dosing a fuel with a copolymer of ethylene and an alkyl acrylate. Preferably the process further comprises the step of dosing the fuel with a citric acid ester of a monoglyceride.

In one aspect the present invention provides use of a citric acid ester of a monoglyceride, for reducing, preventing or inhibiting cold filter plugging in a diesel engine. Preferably the use further comprises the use of a copolymer of ethylene and an alkyl acrylate for reducing, preventing or inhibiting cold filter plugging in a diesel engine.

In one aspect the present invention provides use of a copolymer of ethylene and an alkyl acrylate, for reducing, preventing or inhibiting cold filter plugging in a diesel engine. Preferably the use further comprises the use of a citric acid ester of a monoglyceride for reducing, preventing or inhibiting cold filter plugging in a diesel engine.

Each of the preferred aspects recited herein in respect of the citric acid ester of a monoglyceride and in respect of the copolymer of ethylene and an alkyl acrylate, apply equally to these further aspects of the invention.

Aspects of the invention are defined in the appended claims.

The present invention will now be described in further detail in the following examples, in which:

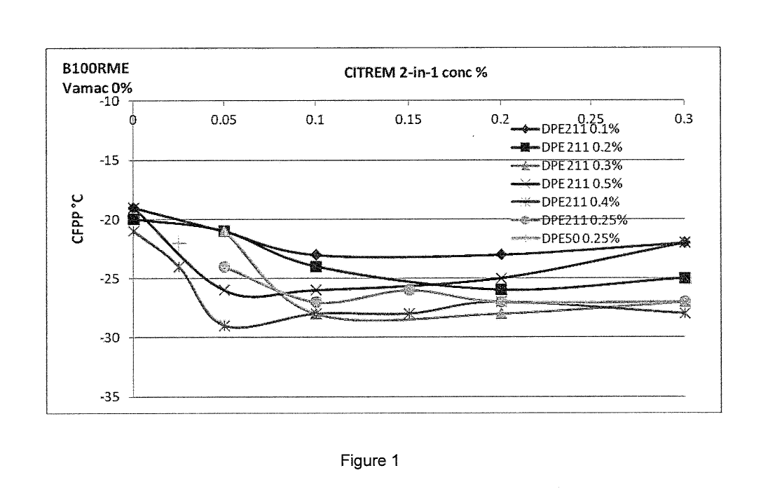

FIG. 1 shows CFPP results in B100 RME depending on concentration of an ester of a polyol and a fatty acid oligomer+a citric acid ester of a monoglyceride.

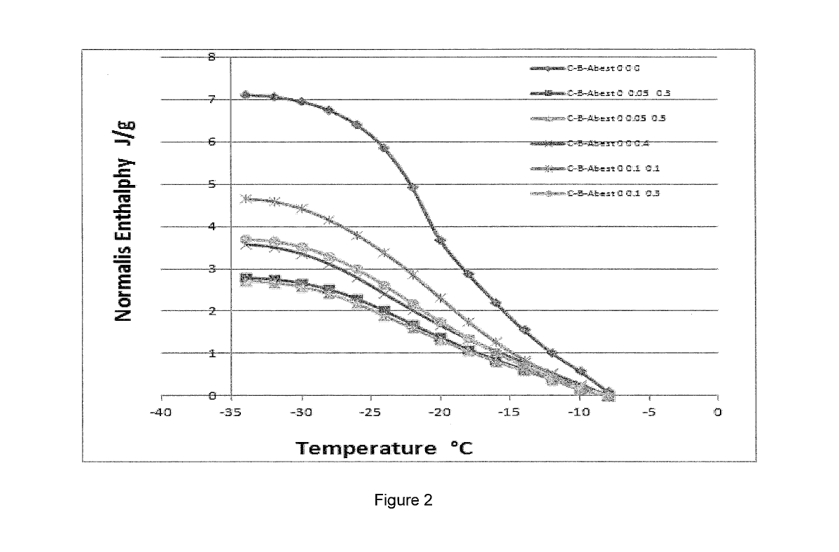

FIG. 2 shows DSC curve showing the solid fat content of B100 RME with an ester of a polyol and a fatty acid oligomer+a citric acid ester of a monoglyceride.

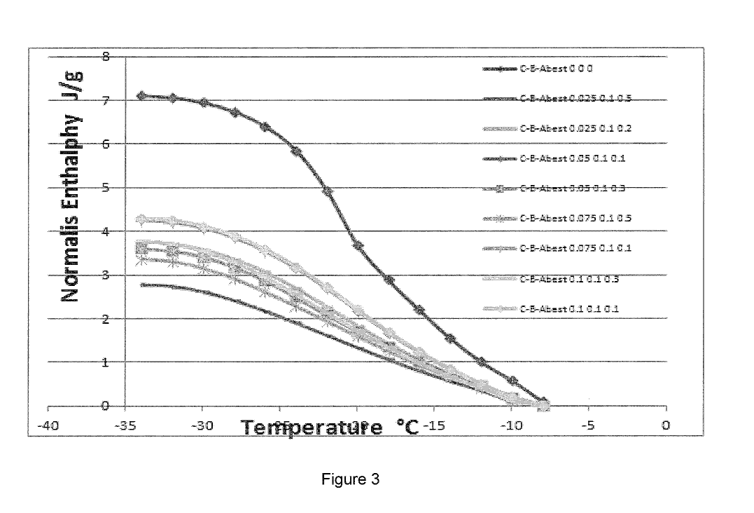

FIG. 3 shows DSC curve showing the solid fat content of B100 RME with an ester of a polyol and a fatty acid oligomer+a copolymer of ethylene and an alkyl acrylate.

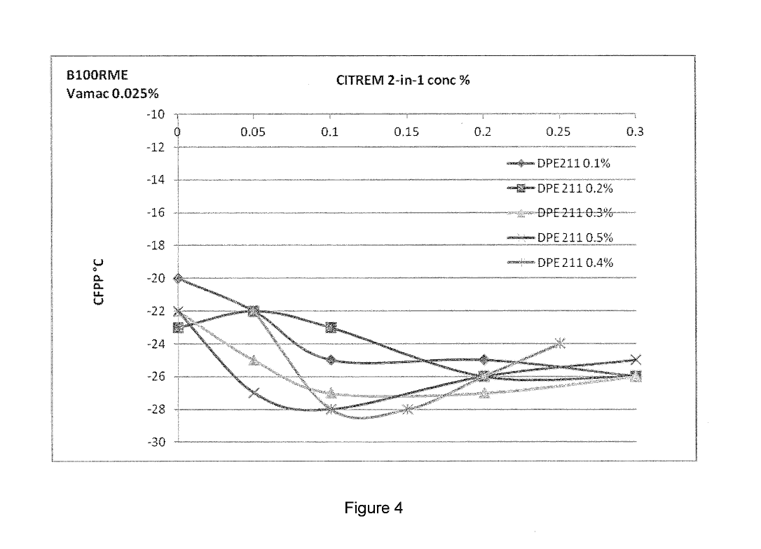

FIG. 4 shows CFPP results in B100 RME depending on concentration of an ester of a polyol and a fatty acid oligomer, +a citric acid ester of a monoglyceride+0.025% a copolymer of ethylene and an alkyl acrylate.

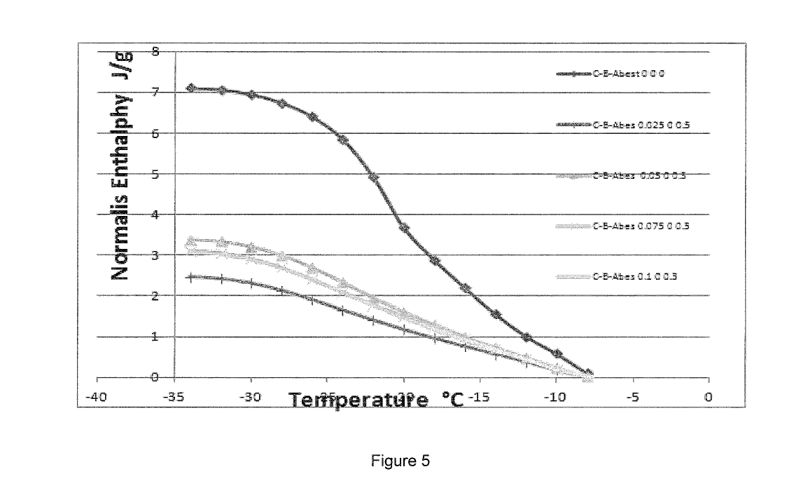

FIG. 5 shows DSC curve showing the solid fat content of B100 RME with an ester of a polyol and a fatty acid oligomer+a citric acid ester of a monoglyceride+a copolymer of ethylene and an alkyl acrylate.

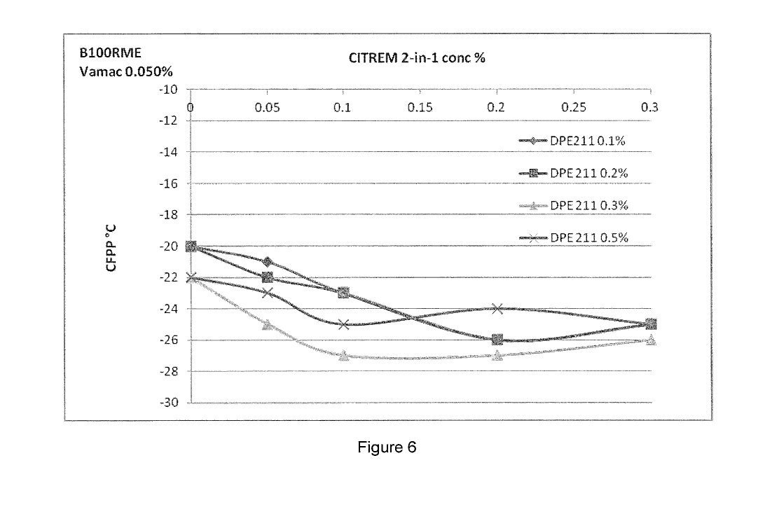

FIG. 6 shows CFPP results in B100 RME depending on concentration of an ester of a polyol and a fatty acid oligomer+a citric acid ester of a monoglyceride+0.05% a copolymer of ethylene and an alkyl acrylate.

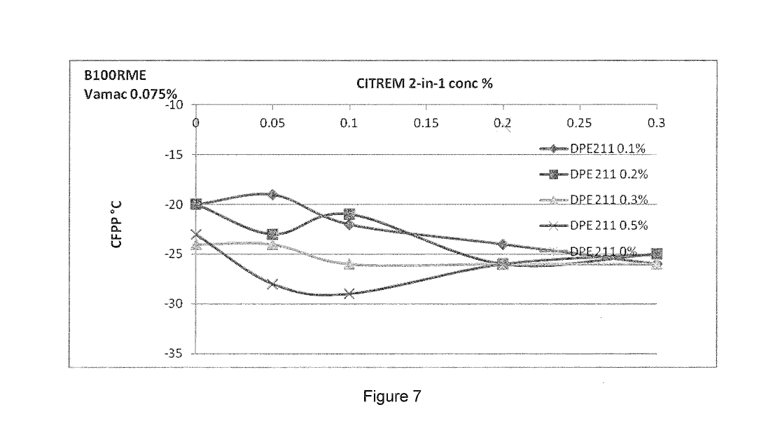

FIG. 7 shows CFPP results in B100 RME depending on concentration of an ester of a polyol and a fatty acid oligomer+a citric acid ester of a monoglyceride+0.075% a copolymer of ethylene and an alkyl acrylate.

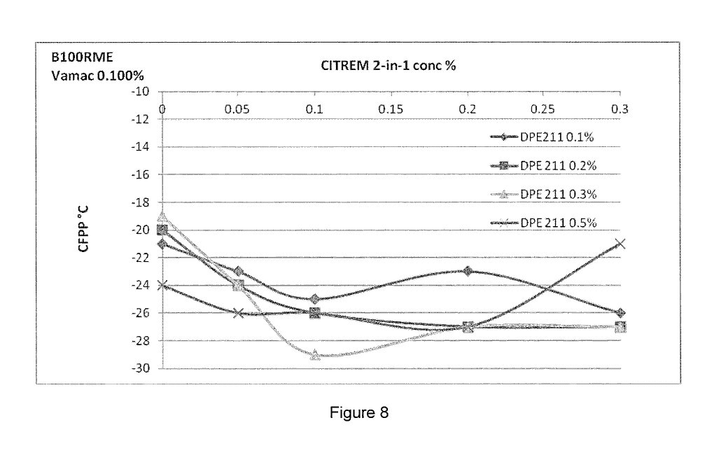

FIG. 8 shows CFPP results in B100 RME depending on concentration of an ester of a polyol and a fatty acid oligomer+a citric acid ester of a monoglyceride+0.10% a copolymer of ethylene and an alkyl acrylate.

### EXAMPLES

In the present Examples, the following abbreviations are used

CFI is a cold flow improver.

CFI A is cold flow improver which is an ester of a polyol and a fatty acid oligomer as described herein.

CFI B is cold flow improver which is a citric acid ester of a monoglyceride as described herein.

CFI C is cold flow improver which is a copolymer of ethylene and an alkyl acrylate as described herein.

CFPP is cold filter plugging point.

OHV is hydroxyl value

PFA is polymerised fatty acid. In this context polymerised provides oligomers and PFA is a fatty acid oligomer as described herein.

DPE is dipentaerythritol.

RA is ricinoleic acid

HSA is 12-hydroxy stearic acid

2IN1 is GRINDSTED® CITREM 2-IN-1

TME is a tallow oil based biodiesel available from DAKA.

SME is a soy bean oil based biodiesel available from Emmelev.

RME is a rapeseed oil based biodiesel available from ADM.

PME is a palm oil based biodiesel available from ADM.

When a biodiesel material incorporates a number, the number denotes the wt. % of biodiesel blended with petro diesel, where the petro diesel makes up the remainder of the diesel blend. For example, B100 RME means that this is solely rapeseed oil based biodiesel and no petro diesel is added, whereas B7 SME is a blend of petro diesel and biodiesel where the biodiesel is soy bean oil based biodiesel and corresponds to 7 wt % of the blend.

### Example 1

### Synthesis of an Ester of a Polyol and a Fatty Acid Oligomer (CFI A)

### Synthesis of PFA: (Polymerisation of Fatty Acid Either as a Mix of Two Different FA or a Single FA Type)

For the preparation of the fatty acid oligomer, the following general preparation process is used.

The fatty acids are slowly heated up to 90° C. protected under nitrogen-cover, reduce pressure to 50 mb, temperature is slowly raised to a reaction temperature of 205° C. The polymerisation process continues until an acid value of 40-45 mg KOH/g is reached (processing time approx. 7-8 hours). See table 6 for examples. Description of analysis method is found in Appendix 1.

### Synthesis of Polyol: (Polymerisation of Glycerol or Like Molecules)

Glycerol is heated up to approx. 220° C. protected under Nitrogen-cover, reduce pressure slowly to 250-200 mb, temperature is raised to reaction temperature of 235° C. The polymerisation process continues until a hydroxyl value (OHV) of 800-1200 is reached (processing time approx. 15 hours). The polyol product will have a certain polyol distribution.

In an alternative, a polyol may be purchased as a commercial product.

A number of polyol distributions from both polyols prepared as described and purchased polyols are given in table 5. A description of analysis method is found in Appendix 2.

### Synthesis of CFI A: (Esterification Between PFA and Polyol)

The mixture of PFA+Polyol+NaOH aq. (50%), wherein the PFA+Polyol are present in the amount described in Table 1 and the NaOH is present in an amount of approximately one twentieth that of the polyol is slowly heated up to 90° C. protected under nitrogen-cover, reduce pressure to 50 mb, temperature is slowly raised to reaction temperature of 205° C. The esterification process continues until an acid value of <2 mg KOH/g is reached (processing time approx. 7-8 hours). See table 2, 3 and 4 for examples of the physical parameters that characterize CFI A. Descriptions of analysis methods are found in Appendix 3, 4, 5 & 6.

Several batches are made of this product A.

In a preferred aspect the polyol in CFI A is dipentaerythritol (DPE). If not stated otherwise the DPE of the Examples (which is available as a commercial product) is 85% pure.

In a preferred aspect the fatty acid polymer reactant in CFI A is synthesized from 80 wt % 12-hydroxystearic acid and 20 wt % ricinoleic acid both derived from castor oil.

### Analysis of CFI A:

A number of CFI A products are synthesised and analysed. The details of the analysis are given below

TABLE 1
CFI A analyses

| | | | Polyol %/ |
|---|---|---|---|
| CFI A | | Polymerised | FA % |
| Product No. | Polyol backbone | Fatty Acid | (wt. %) |
| 2526/150 | Hexaglycerol | 100% HSA | 5.1/94.9 |
| 2526/186 | Hexaglycerol | 90% HSA, 10% RA | 4.8/95.2 |
| 2680/025 | Hexaglycerol | 80% HSA, 20% RA | 4.8/95.2 |
| 2526/173 | Hexaglycerol | 80% HSA, 20% RA | 4.8/95.2 |
| 2526/209 | Hexaglycerol | 80% HSA, 20% RA | 4.9/95.1 |
| 2526/172 | Hexaglycerol | 60% HSA, 40% RA | 4.9/95.1 |
| 2526/147 | Hexaglycerol | 50% HSA, 50% RA | 4.3/95.7 |
| 2863/017 | Hexaglycerol | 100% RA | 6.2/93.8 |
| 2349/140 | Hexaglycerol | 100% Oleic acid | 9.8/90.2 |
| 2349/141 | Hexaglycerol | 100% Stearic Acid | 9.8/90.2 |
| 2526/185 | Triglycerol | 90% HSA, 10% RA | 4.2/95.8 |
| 2526/191 | Triglycerol | 80% HSA, 20% RA | 4.1/95.9 |
| 2526/187 | Decaglycerol | 90% HSA, 10% RA | 5.8/94.2 |
| 2526/192 | Decaglycerol | 80% HSA, 20% RA | 5.9/94.1 |
| 2461/187 | Glycerol | 80% HSA, 20% RA | 20.0/80.0 |
| 2526/205 | Polyethylenglycol | 80% HSA, 20% RA | 10.8/89.2 |
| 2526/194 | Hexandiol | 80% HSA, 20% RA | 4.9/95.1 |
| 2525/204 | 50 wt % Hexandiol | 80% HSA, 20% RA | 4.9/95.1 |
| | 50 wt % Glycerol | | |
| 2680/015 | Erythritol | 80% HSA, 20% RA | 4.4/95.6 |
| 2526/197 | Pentaerythritol | 80% HSA, 20% RA | 4.4/95.6 |
| 2526/195 | 50 wt % Glycerol | 80% HSA, 20% RA | 4.4/95.6 |
| | 50 wt % Pentaerythritol | | |
| 2526/198 | 50 wt % DPE | 80% HSA, 20% RA | 4.4/95.6 |
| | 50 wt % glycerol | | |
| 2680/064 | 75 wt % DPE | 80% HSA, 20% RA | 4.4/95.6 |
| | 25 wt % glycerol | | |
| 2680/041 | DPE | 100% HSA | 4.3/95.7 |
| 2526/211 | DPE | 80% HSA, 20% RA | 4.4/95.6 |
| 2680/018 | DPE | 80% HSA, 20% RA | 4.4/95.6 |
| 2680/050 | DPE | 80% HSA, 20% RA | 4.3/95.7 |
| 2680/051 | DPE 90% purity | 80% HSA, 20% RA | 4.3/95.7 |
| 2680/044 | DPE | 50% HSA, 50% RA | 4.3/95.7 |
| 2680/043 | DPE | 100% RA | 4.3/95.7 |

HSA: 12-hydroxystearic acid
RA: ricinoleic acid

### Analysis of Polyol Distribution

The polyol distribution of the following products is analysed.

TABLE 2a

| | | | Polyol %/ |
|---|---|---|---|
| CFI A | | Polymerised | FA % |
| Product No. | Polyol backbone | Fatty Acid | (wt. %). |
| 2863/017 | Hexaglycerol | 100% RA | 6.2/93.8 |
| 2526/191 | Triglycerol | 80% HSA, 20% RA | 4.1/95.9 |
| 2680/015 | Erythritol | 80% HSA, 20% RA | 4.4/95.6 |
| 2526/197 | Pentaerythritol | 80% HSA, 20% RA | 4.4/95.6 |
| 2526/211 | DPE | 80% HSA, 20% RA | 4.4/95.6 |

The results are given in Tables 2b and 2c.

TABLE 2b
Polyol distribution in CFI A

| Polyol | | |
|---|---|---|
| distribution (GC)* | 2863/017 | 2526/191 |
| Glycerol | 4.15 | 2.25 |
| Cy-diglycerol | 7.35 | 0.15 |
| Diglycerol | 8.33 | 17.97 |
| Cy-triglycerol | 4.03 | 1.72 |
| Triglycerol | 9.96 | 28.83 |
| Cy-tetraglycerol | 3.36 | 3.01 |
| Tetraglycerol | 8.09 | 7.79 |
| Cy-pentaglycerol | 4.05 | 1.06 |
| Pentaglycerol | 6.62 | 3.51 |
| Hexaglycerol | 8.26 | 1.30 |
| Heptaglycerol | 7.72 | 0.21 |
| Octaglycerol | 5.54 | 0.00 |
| Nonaglycerol | 3.85 | 0.00 |
| Decaglycerol | 2.62 | 0.00 |

*wt. % based on total mass of polyols

TABLE 2c
Total Polyol content of CFI A

| | 2863/017 | 2526/191 | 2680/015 | 2526/197 | 2526/211 |
|---|---|---|---|---|---|
| Total Polyol | 6.4 | 5.4 | 4.8 | 5.4 | 4.9 |
| Content | | | | | |
| (wt. %) | | | | | |

### Chemical Characteristic of CFI A

The acid value, saponification value, hydroxyl value and average fatty acid chain length is determined for the following CFI A materials.

TABLE 3a

| | | | Polyol %/ |
|---|---|---|---|
| CFI A | | Polymerised | FA % |
| Product No. | Polyol backbone | Fatty Acid | (wt. %) |
| 2526/173 | Hexaglycerol | 80% HSA, 20% RA | 4.8/95.2 |
| 2863/017 | Hexaglycerol | 100% RA | 6.2/93.8 |
| 2349/140 | Hexaglycerol | 100% Oleic acid | 9.8/90.2 |
| 2349/141 | Hexaglycerol | 100% Stearic Acid | 9.8/90.2 |
| 2526/191 | Triglycerol | 80% HSA, 20% RA | 4.1/95.9 |
| 2680/015 | Erythritol | 80% HSA, 20% RA | 4.4/95.6 |
| 2526/197 | Pentaerythritol | 80% HSA, 20% RA | 4.4/95.6 |
| 2680/041 | DPE | 100% HSA | 4.3/95.7 |
| 2526/211 | DPE | 80% HSA, 20% RA | 4.4/95.6 |
| 2680/044 | DPE | 50% HSA, 50% RA | 4.3/95.7 |
| 2680/043 | DPE | 100% RA | 4.3/95.7 |

The results are given in Table 3b.

TABLE 3b
Chemical characteristic of CFI A

| | Acid | Saponification | Hydroxyl | Average |
|---|---|---|---|---|
| | Value | Value | Value | number of fatty |
| | (AV) | (SV) | (OHV) | acid chains |
| 2526/173 | — | — | — | 4.04 |
| 2863/017 | — | 187 | — | 4.09 |
| 2349/140 | 5.1 | 184 | — | 2.66 |
| 2349/141 | 5.4 | 183 | — | 2.62 |
| 2526/191 | 3.1 | 187 | 42.5 | 4.10 |
| 2680/015 | 0.5 | 189 | 61.0 | 4.98 |
| 2526/197 | — | — | — | 4.32 |
| 2680/041 | 2.3 | 187 | — | 3.81 |
| 2526/211 | 2.5 | 188 | 47.8 | 4.48 |
| 2680/044 | 2.5 | 187 | — | 3.70 |
| 2680/043 | 2.5 | — | — | 4.12 |

### Fatty Acid Distribution

The fatty acid distribution is determined for the following materials.

TABLE 4a

| | | | Polyol %/ |
|---|---|---|---|
| CFI A | | Polymerised | FA % |
| Product No. | Polyol backbone | Fatty Acid | (wt. %)% |
| 2863/017 | Hexaglycerol | 100% RA | 6.2/93.8 |
| 2349/140 | Hexaglycerol | 100% Oleic acid | 9.8/90.2 |
| 2349/141 | Hexaglycerol | 100% Stearic Acid | 9.8/90.2 |
| 2526/191 | Triglycerol | 80% HSA, 20% RA | 4.1/95.9 |
| 2680/015 | Erythritol | 80% HSA, 20% RA | 4.4/95.6 |
| 2526/197 | Pentaerythritol | 80% HSA, 20% RA | 4.4/95.6 |
| 2680/041 | DPE | 100% HSA | 4.3/95.7 |
| 2526/211 | DPE | 80% HSA, 20% RA | 4.4/95.6 |
| 2680/044 | DPE | 50% HSA, 50% RA | 4.3/95.7 |
| 2680/043 | DPE | 100% RA | 4.3/95.7 |

The results are given in Table 4b.

TABLE 4b
Fatty acid distribution in PFA measured in CFI A

| | 2863/ | 2349/ | 2349/ | 2526/ | 2680/ | 2526/ | 2680/ | 2526/ | 2680/ | 2680/ |
|---|---|---|---|---|---|---|---|---|---|---|
| Methyl ester | 017 | 140 | 141 | 191 | 015 | 197 | 041 | 211 | 044 | 043 |
| C14 | 0.1 | 0.0 | 0.0 | 0.0 | 0.0 | 0.0 | 0.0 | 0.0 | 0.0 | 0.0 |
| C16 | 5.9 | 1.1 | 10.3 | 1.0 | 1.0 | 1.1 | 1.0 | 1.1 | 1.1 | 1.0 |
| C16:1 | 0.1 | 0.0 | 0.0 | 0.0 | 0.0 | 0.0 | 0.0 | 0.0 | 0.0 | 0.0 |
| C17 | 0.0 | 0.0 | 0.0 | 0.0 | 0.0 | 0.0 | 0.0 | 0.0 | 0.0 | 0.0 |
| C18 | 5.4 | 1.4 | 12.3 | 6.9 | 7.0 | 6.9 | 8.3 | 7.0 | 5.0 | 1.2 |
| C18:1 | 3.9 | 21.5 | 2.5 | 2.4 | 2.0 | 2.0 | 1.9 | 2.7 | 2.8 | 2.9 |
| C18:2 | 5.1 | 4.5 | 3.2 | 0.9 | 0.9 | 0.9 | 0.1 | 1.0 | 2.2 | 4.0 |
| C18:3 | 0.0 | 0.0 | 0.0 | 0.0 | 0.0 | 0.0 | 0.0 | 0.0 | 0.0 | 0.0 |
| C20 | 0.1 | 0.1 | 0.1 | 0.3 | 0.3 | 0.3 | 0.4 | 0.3 | 0.2 | 0.0 |
| C20:1 | 0.0 | 0.0 | 0.0 | 0.0 | 0.0 | 0.0 | 0.0 | 0.0 | 0.0 | 0.0 |
| 12-ketoacid | 0.2 | 0.1 | 0.2 | 0.8 | 0.8 | 0.9 | 1.1 | 0.8 | 0.5 | 0.3 |
| C18 | 76.1 | 69.4 | 69.0 | 86.8 | 85.7 | 86.9 | 86.6 | 85.0 | 86.4 | 86.9 |
| hydroxyacid | | | | | | | | | | |
| Unknown | 3.2 | 1.9 | 2.4 | 1.0 | 2.3 | 1.0 | 0.5 | 2.2 | 1.9 | 3.7 |

TABLE 5
Examples of polyol distribution & characteristic in
the CFI A raw material, the polyol backbone, utilised
in the synthesis of the products listed in Table 1

| | Triglycerol | I/Hexaglycerol | |
|---|---|---|---|
| Analysis | (062365) | (161855) | Decaglycerol |
| Glycerol | 0.1 | 5.2 | 3.3 |
| Cy-diglycerol | 0.0 | 9.0 | 11.8 |
| Diglycerol | 27.0 | 9.7 | 6.7 |
| Cy-triglycerol | 2.5 | 5.7 | 7.4 |
| Triglycerol | 44.2 | 10.3 | 8.1 |
| Cy-tetraglycerol | 4.1 | 4.0 | 5.0 |
| Tetraglycerol | 12.4 | 8.5 | 7.2 |
| Cy-pentaglycerol | 1.6 | 4.1 | 5.3 |
| Pentaglycerol | 4.2 | 6.8 | 6.1 |
| Hexaglycerol | 2.5 | 9.2 | 9.3 |
| Heptaglycerol | 1.2 | 8.5 | 9.8 |
| Octaglycerol | 0.0 | 7.0 | 8.5 |
| Nonaglycerol | 0.0 | 5.4 | 7.3 |
| Decaglycerol | 0.0 | 3.6 | 4.3 |
| Undecaglycerol | 0.0 | 3.1 | 0.0 |
| Sum | 100.0 | 100.0 | 100.0 |
| OHV | 1127 | 962 | 882 |

### Acid Value of PFAs

The acid values for the following PFAs used to prepare the CFI A materials are measured.

TABLE 5a

| | | | Polyol %/ |
|---|---|---|---|
| CFI A | | Polymerised | FA % |
| Product No. | Polyol backbone | Fatty Acid | (wt. %) |
| 2526/173 | Hexaglycerol | 80% HSA, 20% RA | 4.8/95.2 |
| 2863/017 | Hexaglycerol | 100% RA | 6.2/93.8 |
| 2349/140 | Hexaglycerol | 100% Oleic acid | 9.8/90.2 |
| 2349/141 | Hexaglycerol | 100% Stearic Acid | 9.8/90.2 |
| 2526/191 | Triglycerol | 80% HSA, 20% RA | 4.1/95.9 |
| 2680/015 | Erythritol | 80% HSA, 20% RA | 4.4/95.6 |
| 2526/197 | Pentaerythritol | 80% HSA, 20% RA | 4.4/95.6 |
| 2680/041 | DPE | 100% HSA | 4.3/95.7 |
| 2526/211 | DPE | 80% HSA, 20% RA | 4.4/95.6 |
| 2680/044 | DPE | 50% HSA, 50% RA | 4.3/95.7 |
| 2680/043 | DPE | 100% RA | 4.3/95.7 |
| 2653/058 | DPE | 80% HSA, 20% RA | 5.9/94.1 |
| 2697/114 | DPE | 80% HSA, 20% RA | 8.9/91.1 |
| 2697/144 | DPE | 80% HSA, 20% RA | 3.5/96.5 |
| 2680/064 | 75 wt % DPE | 80% HSA, 20% RA | 4.4/95.6 |
| | 25 wt % glycerol | | |

The results are given in Table 6.

TABLE 6
Physical characteristic in the CFI A raw
material PFA (polymerized fatty acid)

| | AV of PFA |
|---|---|
| 2526/173 | 43.1 |
| 2863/017 | 43.0 |
| 2349/140 | 80.0 |
| 2349/141 | 78.8 |
| 2526/191 | 43.1 |
| 2680/015 | 43.9 |
| 2526/197 | 43.1 |
| 2680/041 | 41.4 |
| 2526/211 | 45.3 |
| 2680/044 | 42.6 |
| 2680/043 | 42.0 |
| 2653/058 | 42.6 |
| 2697/114 | 43.8 |
| 2697/144 | 42.6 |
| 2680/064 | 42.5 |

Acid value is an accurate indirect measure of the degree of polymerisation of the fatty acid in the polymerized fatty acid.

### Example 2

### Synthesis of CFI B

As discussed herein, in one aspect the present invention provides a citric acid ester of a monoglyceride which may be used as a CFI. Citric acid esters of monoglyceride are typically referred to as CITREMs.

In the present examples, CFI B is a cold flow improver which is a citric acid ester of a monoglyceride as described herein.

In the present examples the CFI Bs are GRINDSTED® CITREM 2-IN-1 (a number of different batches), Citrem LR10 extra and Citrem SP70. Each of which is a citric acid ester of a monoglyceride available from DuPont (formerly Danisco A/S, Denmark). The Citrems are prepared from monoglycerides derived from a variety of oil sources. The oil sources for the tested Citrems are given below. Different lab batches of GRINDSTED® CITREM 2-IN-1 are denoted by batch numbers, such as 2447/085 and 2447/088.

### Analysis of CFI B:

TABLE 7
CFI B range and compositions & characteristic

| | | Total mono- | Citric acid | | |
|---|---|---|---|---|---|
| | | glyceride | content in | | |
| CFI B | Oil source | content* | reaction* | AV | SV |
| Citrem 2IN1 | 80% High oleic | 68% | 17.9% | 21.7 | 298 |
| | sunflower | | | | |
| | oil + 20% | | | | |
| | sunflower oil | | | | |
| Citrem 2IN1 - | 80% High oleic | 68% | 17.9% | 18.0 | 301 |
| 2447/085 | sunflower | | | | |
| | oil + 20% | | | | |
| | sunflower oil | | | | |
| Citrem 2IN1 - | 80% High oleic | 68% | 17.9% | 18.4 | 299 |
| 2447/088 | sunflower | | | | |
| | oil + 20% | | | | |
| | sunflower oil | | | | |
| Citrem 2IN1 - | High oleic | >95% | 20.9% | 21.7 | 321 |
| 2447/084 | sunflower oil | | | | |
| Citrem 2IN1 - | Sunflower oil | 68% | 17.9% | 19.0 | 300 |
| 2447/087 | | | | | |
| Citrem 2IN1 - | Sunflower oil | >95% | 20.9% | 20.1 | 320 |
| 2447/086 | | | | | |
| Citrem 2IN1 - | Rapeseed oil | 68% | 17.9% | 19.2 | 292 |
| 2447/089 | | | | | |
| Citrem LR10 | High oleic | 86% | 14.4% | 25.5 | 264 |
| extra | sunflower oil | | | | |
| Citrem SP70 | Sunflower oil | 87% | 13.0% | 16.0 | 258 |

*Values are based on calculation of the raw material content used in the reaction.

### Example 3

### Synthesis/Analysis of CFI C

As discussed herein, in one aspect the present invention provides a copolymer of ethylene and an alkyl acrylate which may be used as a CFI.

In the present examples, CFI C is cold flow improver which is a copolymer of ethylene and an alkyl acrylate.

### Synthesis of CFI C:

This polymer is a commercial DuPont product, Vamac DP. Suitable CFI C polymers may also be prepared in accordance with the teachings of U.S. Pat. No. 7,544,757.

### Analysis of CFI C:

TABLE 8
CFI C range and compositions & characteristic.

| | | | Melt | Acid |
|---|---|---|---|---|
| | | Copolymers | Index | content |
| CFI C | MW range | constituents | (MI) | Index |
| Vamac DP | 40,000-65,000 | 1. Methyl acrylate | 2-12 g/ | 0 |
| | | 2. Ethylene | 10 min | |
| Vamac DP | 40,000-65,000 | 1. Methyl acrylate | 2-12 g/ | 0 |
| 5411040008-046 | | 2. Ethylene | 10 min | |
| Vamac 3038 To | 40,000-65,000 | 1. Methyl acrylate | 1-6/ | 200 |
| 11031027 | | 2. Ethylene | 10 min | |
| | | 3. Acidic cure | | |
| | | compound | | |
| Vamac GLS lot | 40,000-65,000 | 1. Methyl acrylate | 1-6/ | 100 |
| 5411060021 | | 2. Ethylene | 10 min | |
| | | 3. Acidic cure | | |
| | | compound | | |
| Vamac VCD | 40,000-65,000 | 1. Methyl acrylate | 2-12 g/ | None |
| 6200 | | 2. Ethylene | 10 min | |

The acidic cure compound is a compound of the formula (R)z—C═O(—OH)

### Example 4

### Application Tests

The following CFIs are tested: A, A+B, A+C and A+B+C.

In the following there will be examples of CFPP results divided in to these groups:

Sample A is categorized into three groups to assist in the assessment of the classes which are being tested. The following denotations are used:

CFI A Ery=esters of PFA+pentaerythritol derivatives

CR A Hex=esters of PFA+Hexaglycerol

CFI A Poly=esters of PFA+other Polyols

CFI A Ery is referred to in the figures as Abest or Abes

### Application in Biofuels—CFI A Ery

A range of esters of PFA+pentaerythritol derivatives dosed in biodiesel B100 RME are tested. The cold filter plugging point is measured and can be compared against the control diesel containing no additive.

TABLE 9
B100 RME

| CFI A Ery | Dosage w/v % | CFPP (° C.) * |
|---|---|---|
| None (control) | — | −14 |
| 2680/015 | 0.2 | −18 |
| | 0.5 | −15 |
| | 1.0 | −16 |
| | 2.0 | −15 |
| 2526/197 | 0.2 | −11 |
| | 0.5 | −15 |
| | 1.0 | −16 |
| | 2.0 | −17 |
| 2526/195 | 0.2 | −15 |
| | 0.5 | −14 |
| | 1.0 | −13 |
| | 2.0 | −12 |
| 2526/211 | 0.05 | −16 |
| | 0.1 | −19 |
| | 0.2 | −19 |
| | 0.25 | −19 |
| | 0.3 | −20 |
| | 0.5 | −19 |
| | 0.75 | −17 |
| | 1.0 | −16 |
| | 2.0 | −19 |
| 2526/198 | 0.2 | −15 |
| | 0.5 | −18 |
| | 1.0 | −15 |
| | 2.0 | −13 |
| 2680/041 | 0.3 | −19 |
| 2680/043 | 0.3 | −15 |
| 2680/044 | 0.3 | −19 |
| 2680/018 | 0.3 | −22 |
| 2680/050 | 0.3 | −23 |
| 2680/051 | 0.3 | −19 |

* The equipment that is used to determine CFPP has an accuracy of +/−2° C.

A preferred ester of PFA+pentaerythritol derivative is then dosed in bio diesel B100 TME and tested. The cold filter plugging point is measured and can be compared against the control diesel containing no additive.

TABLE 10
B100 TME

| CFI A Ery | Dosage w/v % | CFPP (° C.) |
|---|---|---|
| None (control) | — | 10 |
| 2526/211 | 0.05 | 11 |
| | 0.1 | 11 |
| | 0.2 | 10 |
| | 0.5 | 10 |
| | 1.0 | 10 |
| | 2.0 | 10 |

### Application in Biofuels CFI A Ery+CFI B

Preferred ester of PFA+pentaerythritol derivatives in combination with preferred Citrems are dosed in bio diesel B100 RME and tested. The cold filter plugging point is measured and can be compared against the control diesel containing no additive. B100 RME is a blend of methyl esters derived from a transesterification of rapeseed oil with methanol. B100 means that this is solely biodiesel and no petro diesel is added. If instead B7 where mentioned it is a blend of petro diesel and biodiesel where the biodiesel corresponds to 7% of the blend.

TABLE 11
CFI A Ery + CFI B in B100 RME

| | Dosage | | Dosage | |
|---|---|---|---|---|
| CFI A Ery | w/v % | CFI B | w/v % | CFPP (° C.) |
| None (control) | — | None (control) | — | −14 |
| 2680/015 | 0.1 | 2IN1 | 0.2 | −13 |
| | 0.3 | 2IN1 | 0.2 | −17 |
| 2526/197 | 0.1 | 2IN1 | 0.2 | −13 |
| | 0.3 | 2IN1 | 0.2 | −14 |
| 2526/195 | 0.1 | 2IN1 | 0.2 | −13 |
| | 0.3 | 2IN1 | 0.2 | −16 |
| 2526/211 | 0.1 | 2IN1 | 0.05 | −21 |
| | 0.2 | 2IN1 | 0.05 | −21 |
| | 0.3 | 2IN1 | 0.05 | −21 |
| | 0.5 | 2IN1 | 0.05 | −26 |
| | 0.1 | 2IN1 | 0.1 | −23 |
| | 0.2 | 2IN1 | 0.1 | −24 |
| | 0.3 | 2IN1 | 0.1 | −28 |
| | 0.3 | 2IN1 | 0.1 | −26 |
| | 0.5 | 2IN1 | 0.1 | −26 |
| | 0.1 | 2IN1 | 0.2 | −23 |
| | 0.2 | 2IN1 | 0.2 | −26 |
| | 0.3 | 2IN1 | 0.2 | −28 |
| | 0.5 | 2IN1 | 0.2 | −25 |
| | 0.1 | 2IN1 | 0.3 | −22 |
| | 0.2 | 2IN1 | 0.3 | −25 |
| | 0.3 | 2IN1 | 0.3 | −27 |
| | 0.5 | 2IN1 | 0.3 | −22 |
| | 0.4 | 2IN1 | 0.05 | −29 |
| | 0.4 | 2IN1 | 0.1 | −28 |
| | 0.4 | 2IN1 | 0.15 | −28 |
| | 0.4 | 2IN1 | 0.2 | −27 |
| | 0.4 | 2IN1 | 0.3 | −28 |
| | 0.25 | 2IN1 | 0.05 | −24 |
| | 0.25 | 2IN1 | 0.1 | −27 |
| | 0.25 | 2IN1 | 0.15 | −26 |
| | 0.25 | 2IN1 | 0.2 | −27 |
| | 0.25 | 2IN1 | 0.3 | −27 |
| | 0.25 | 2IN1 | 0.025 | −22 |
| | 0.3 | LR10 | 0.15 | −25 |
| | 0.3 | LR10 | 0.25 | −27 |
| | 0.3 | LR10 | 0.3 | −28 |
| | 0.3 | LR10 | 0.35 | −28 |
| | 0.3 | LR10 | 0.375 | −26 |
| | 0.3 | LR10 | 0.4 | −29 |
| | 0.3 | LR10 | 0.45 | −28 |
| | 0.3 | LR10 | 0.5 | −25 |
| | 0.3 | SP70 | 0.15 | −21 |
| | 0.3 | SP70 | 0.2 | −19 |
| | 0.3 | 2447/084 | 0.2 | −27 |
| | 0.3 | 2447/085 | 0.2 | −27 |
| | 0.3 | 2447/087 | 0.2 | −27 |
| | 0.3 | 2447/088 | 0.2 | −26 |
| | 0.3 | 2447/089 | 0.2 | −23 |
| 2526/198 | 0.25 | 2IN1 | 0.025 | −20 |
| | 0.3 | 2IN1 | 0.15 | −26 |
| | 0.4 | 2IN1 | 0.15 | −26 |
| | 0.4 | 2IN1 | 0.2 | −26 |
| | 0.5 | 2IN1 | 0.2 | −25 |
| | 0.1 | 2IN1 | 0.2 | −19 |
| | 0.25 | 2IN1 | 0.025 | −20 |
| | 0.3 | 2IN1 | 0.2 | −26 |
| | 0.3 | 2447/088 | 0.2 | −25 |
| 2680/041 | 0.3 | 2IN1 | 0.2 | −25 |
| 2680/043 | 0.3 | 2IN1 | 0.2 | −12 |
| 2680/044 | 0.3 | 2IN1 | 0.2 | −23 |
| | 0.3 | 2447/088 | 0.2 | −24 |
| 2680/018 | 0.3 | 2IN1 | 0.2 | −28 |
| 2680/050 | 0.3 | 2IN1 | 0.2 | −30 |
| | 0.3 | 2IN1 | 0.15 | −28 |
| | 0.3 | 2447/088 | 0.2 | −29 |
| | 0.25 | 2IN1 | 0.025 | −21 |
| 2680/051 | 0.3 | 2IN1 | 0.2 | −28 |
| 2680/064 | 0.3 | 2IN1 | 0.2 | −29 |
| 2697/114 (25%) | 0.3 | 2IN1 | 0.2 | −25 |
| 2653/057 (50%) | 0.3 | 2IN1 | 0.2 | −24 |
| 2653/058 (75%) | 0.3 | 2IN1 | 0.2 | −27 |
| 2697/116 (125%) | 0.3 | 2IN1 | 0.2 | −29 |

The combinations of (i) ester of PFA+pentaerythritol derivatives and (ii) Citrems are then dosed in biodiesel blends and tested. The cold filter plugging point is measured and can be compared against the control diesel containing no additive. TME is a tallow oil biodiesel available from DAKA. SME is a soy bean oil based biodiesel available from Emmelev. RME is a rapeseed oil based biodiesel available from ADM. PME is a palm oil based biodiesel.

TABLE 12
B100 TME - Blends of different biodiesel

| | | Dosage | | Dosage | CFPP |
|---|---|---|---|---|---|
| Biodiesel | CFI A Ery | w/v % | CFI B | w/v % | (° C.) |
| TME | None (control) | — | None (control) | — | 10 |
| | 2526/211 | 0.3 | 2IN1 | 0.2 | 10 |
| | | 1.2 | | 1.0 | 9 |
| | | 3.0 | | 2.0 | 10 |
| | | 0.35 | LR10 | 0.4 | 10 |
| | | 3.0 | | 3.5 | 9 |
| 80:20 | None (control) | — | None (control) | — | 8 |
| (wt. %) | 2526/211 | 0.3 | 2IN1 | 0.2 | 8 |
| TME:SME | | | | | |
| 75:25 | None (control) | — | None (control) | — | −10 |
| (wt. %) | 2526/211 | 0.3 | 2IN1 | 0.2 | −19 |
| RME:SME | | | | | |
| 80:20 | None (control) | — | None (control) | — | −9 |
| (wt. %) | 2526/211 | 0.3 | 2IN1 | 0.2 | −12 |
| RME:SME | | | | | |
| 90:10 | None (control) | — | None (control) | — | −10 |
| (wt. %) | 2526/211 | 0.3 | 2IN1 | 0.15 | −14 |
| RME:PME | | | | | |
| 95:5 | None (control) | — | None (control) | — | −9 |
| (wt. %) | 2526/211 | 0.3 | 2IN1 | 0.2 | −13 |
| RME:PME | | | | | |
| 60:40 | None (control) | — | None (control) | — | 6 |
| (wt. %) | 2526/211 | 0.3 | 2IN1 | 0.15 | 2 |
| SME:TME | | | | | |

CFI A 2526/211 is then tested with each of 2IN1 and LR10 in biodiesel B7 RME. B7 RME is a rape seed biodiesel containing 7% biodiesel and 93% petro diesel.

TABLE 13
B7 RME The dosage w/v % in the table is based on the biodiesel
content in the fuel (i.e. in the total fuel 0.021 w/v %)

| | Dosage | | Dosage | |
|---|---|---|---|---|
| CFI A Ery | w/v % | CFI B | w/v % | CFPP (° C.) |
| None (control) | | — | — | −29 |
| 2526/211 | 0.3 | 2IN1 | 0.2 | −31 |
| | 0.3 | | 0.15 | −34 |
| | 0.3 | | 0.1 | −31 |
| | 0.3 | LR10 | 0.375 | −29 |

CFI A 2526/211 is then tested with 2IN1 in a number of biodiesel blends.

TABLE 14
B10 XME - Blends of different biodiesel The dosage w/v % in the
table is based on the biodiesel content in the fuel (ie. in the
total fuel CFI A Ery 0.03 w/v % and CFI B 0.015 w/v %)

| Biodiesel | | Dosage | | Dosage | CFPP |
|---|---|---|---|---|---|
| blend | CFI A Ery | w/v % | CFI B | w/v % | (° C.) |
| 75:25 | None (control) | — | None (control) | — | −28 |
| RME:SME | 2526/211 | 0.3 | 2IN1 | 0.15 | −26 |
| 90:10 | None (control) | — | None (control) | — | −27 |
| RME:PME | 2526/211 | 0.3 | 2IN1 | 0.15 | −29 |
| 60:40 | None (control) | — | None (control) | — | −18 |
| SME:PME | 2526/211 | 0.3 | 2IN1 | 0.15 | −18 |

TABLE 15
B7 XME - Blends of different biodiesel The dosage w/v % in the
table is based on the biodiesel content in the fuel (ie. in
the total fuel CFI A Ery 0.03 w/v % and CFI B 0.01 w/v %)

| Biodiesel | | Dosage | | Dosage | CFPP |
|---|---|---|---|---|---|
| blend | CFI A Ery | w/v % | CFI B | w/v % | (° C.) |
| 75:25 | None (control) | — | None (control) | — | −29 |
| RME:SME | 2526/211 | 0.3 | 2IN1 | 0.15 | −28 |
| 90:10 | None (control) | — | None (control) | — | −27 |
| RME:PME | 2526/211 | 0.3 | 2IN1 | 0.15 | −29 |
| 60:40 | None (control) | — | None (control) | — | −20 |
| SME:PME | 2526/211 | 0.3 | 2IN1 | 0.15 | −19 |

B10 XME is a biodiesel containing 10% biodiesel and 90% petro diesel

### Application in Biofuels CFI A Ery+CFI C

Preferred ester of PFA+pentaerythritol derivatives in combination with preferred copolymers of ethylene and an alkyl acrylate are dosed in biodiesel B100 RME and tested. The cold filter plugging point is measured and can be compared against the control diesel containing no additive. Vamac DP is a copolymer of ethylene and an alkyl acrylate available from DuPont.

TABLE 16
B100 RME

| | Dosage | | Dosage | |
|---|---|---|---|---|
| CFI A Ery | w/v % | CFI C | w/v % | CFPP (° C.) |
| None (control) | — | None (control) | — | −14 |
| 2526/211 | 0.5 | Vamac DP | 0.01 | −20 |
| | 0.5 | | 0.02 | −21 |
| | 0.5 | | 0.03 | −21 |
| | 0.5 | | 0.04 | −23 |
| | 0.5 | | 0.05 | −23 |
| | 0.5 | | 0.06 | −22 |
| | 0.2 | | 0.075 | −20 |
| | 0.5 | | 0.075 | −25 |
| | 0.1 | | 0.025 | −20 |
| | 0.2 | | 0.025 | −23 |
| | 0.3 | | 0.025 | −22 |
| | 0.5 | | 0.025 | −22 |
| | 0.1 | | 0.05 | −20 |
| | 0.2 | | 0.05 | −20 |
| | 0.3 | | 0.05 | −22 |
| | 0.5 | | 0.05 | −22 |
| | 0.1 | | 0.075 | −20 |
| | 0.2 | | 0.075 | −20 |
| | 0.3 | | 0.075 | −24 |
| | 0.5 | | 0.075 | −23 |
| | 0.1 | | 0.1 | −21 |
| | 0.2 | | 0.1 | −20 |
| | 0.3 | | 0.1 | −19 |
| | 0.5 | | 0.1 | −24 |
| 2680/015 | 0.5 | | 0.075 | −17 |
| 2680/018 | 0.5 | | 0.075 | −23 |
| 2526/197 | 0.5 | | 0.075 | −18 |

CFI A 2526/211 is then tested with Vamac DP in a biodiesel blend.

TABLE 17
B7 RME
The dosage w/v % in the table is based on the biodiesel content in the
fuel (ie. in the total fuel CFI A Ery 0.05 w/v % and CFI C 0.005 w/v %)

| CFI A Ery | Dosage w/v % | CFI C | Dosage w/v % | CFPP (° C.) |
|---|---|---|---|---|
| None | — | — | — | −29 |
| (control) | | | | |
| 2526/211 | 0.5 | Vamac DP | 0.075 | −34 |

### Application in Biofuels CFI A Ery+CFI B+CFI C

Preferred esters of PFA+pentaerythritol derivatives in combination with preferred citric acid esters of monoglycerides and preferred copolymers of ethylene and an alkyl acrylate are dosed in biodiesel B100 RME and tested. The cold filter plugging point is measured and can be compared against the control diesel containing no additive. Vamac 3038, Vamac GLS and Vamac VCD are copolymers of ethylene and an alkyl acrylate available from DuPont.

TABLE 18
CFI A Ery + CFI B + CFI C in B100 RME

| | Dosage | | Dosage | | Dosage | CFPP |
|---|---|---|---|---|---|---|
| CFI A Ery | w/v % | CFI B | w/v % | CFI C | w/v % | (° C.) |
| None | — | None | — | None | — | −14 |
| (control) | | (control) | | (control) | | |
| 2680/041 | 0.3 | 2IN1 | 0.2 | Vamac DP | 0.075 | −25 |
| 2680/043 | 0.3 | | 0.2 | | 0.075 | −13 |
| 2680/044 | 0.3 | | 0.2 | | 0.075 | −25 |
| 2526/211 | 0.1 | | 0.05 | | 0.025 | −22 |
| | 0.2 | | 0.05 | | 0.025 | −22 |
| | 0.3 | | 0.05 | | 0.025 | −24 |
| | 0.5 | | 0.05 | | 0.025 | −27 |
| | 0.1 | | 0.1 | | 0.025 | −25 |
| | 0.2 | | 0.1 | | 0.025 | −23 |
| | 0.3 | | 0.1 | | 0.025 | −26 |
| | 0.5 | | 0.1 | | 0.025 | −28 |
| | 0.1 | | 0.2 | | 0.025 | −25 |
| | 0.2 | | 0.2 | | 0.025 | −26 |
| | 0.3 | | 0.2 | | 0.025 | −28 |
| | 0.5 | | 0.2 | | 0.025 | −26 |
| | 0.1 | | 0.3 | | 0.025 | −26 |
| | 0.2 | | 0.3 | | 0.025 | −26 |
| | 0.3 | | 0.3 | | 0.025 | −26 |
| | 0.5 | | 0.3 | | 0.025 | −25 |
| | 0.4 | | 0.05 | | 0.025 | −22 |
| | 0.4 | | 0.1 | | 0.025 | −28 |
| | 0.4 | | 0.15 | | 0.025 | −28 |
| | 0.3 | | 0.15 | | 0.025 | −28 |
| | 0.4 | | 0.2 | | 0.025 | −26 |
| | 0.4 | | 0.25 | | 0.025 | −24 |
| | 0.1 | | 0.05 | | 0.05 | −21 |
| | 0.2 | | 0.05 | | 0.05 | −22 |
| | 0.3 | | 0.05 | | 0.05 | −25 |
| | 0.5 | | 0.05 | | 0.05 | −23 |
| | 0.1 | | 0.1 | | 0.05 | −23 |
| | 0.2 | | 0.1 | | 0.05 | −23 |
| | 0.3 | | 0.1 | | 0.05 | −27 |
| | 0.5 | | 0.1 | | 0.05 | −25 |
| | 0.1 | | 0.2 | | 0.05 | −26 |
| | 0.2 | | 0.2 | | 0.05 | −26 |
| | 0.3 | | 0.2 | | 0.05 | −27 |
| | 0.5 | | 0.2 | | 0.05 | −24 |
| | 0.1 | | 0.3 | | 0.05 | −25 |
| | 0.2 | | 0.3 | | 0.05 | −25 |
| | 0.3 | | 0.3 | | 0.05 | −26 |
| | 0.5 | | 0.3 | | 0.05 | −25 |
| | 0.1 | | 0.05 | | 0.075 | −19 |
| | 0.2 | | 0.05 | | 0.075 | −23 |
| | 0.3 | | 0.05 | | 0.075 | −24 |
| | 0.5 | | 0.05 | | 0.075 | −28 |
| | 0.1 | | 0.1 | | 0.075 | −22 |
| | 0.2 | | 0.1 | | 0.075 | −21 |
| | 0.3 | | 0.1 | | 0.075 | −26 |
| | 0.5 | | 0.1 | | 0.075 | −29 |
| | 0.1 | | 0.2 | | 0.075 | −24 |
| | 0.2 | | 0.2 | | 0.075 | −26 |
| | 0.3 | | 0.2 | | 0.075 | −26 |
| | 0.5 | | 0.2 | | 0.075 | −26 |
| | 0.1 | | 0.3 | | 0.075 | −26 |
| | 0.2 | | 0.3 | | 0.075 | −25 |
| | 0.3 | | 0.3 | | 0.075 | −26 |
| | 0.5 | | 0.3 | | 0.075 | −25 |
| | 0.1 | | 0.05 | | 0.1 | −23 |
| | 0.2 | | 0.05 | | 0.1 | −24 |
| | 0.3 | | 0.05 | | 0.1 | −24 |
| | 0.5 | | 0.05 | | 0.1 | −26 |
| | 0.1 | | 0.1 | | 0.1 | −25 |
| | 0.2 | | 0.1 | | 0.1 | −26 |
| | 0.3 | | 0.1 | | 0.1 | −29 |
| | 0.5 | | 0.1 | | 0.1 | −26 |
| | 0.1 | | 0.05 | | 0.1 | −25 |
| | 0.2 | | 0.05 | | 0.1 | −23 |
| | 0.3 | | 0.05 | | 0.1 | −29 |
| | 0.5 | | 0.05 | | 0.1 | −28 |
| | 0.1 | | 0.2 | | 0.1 | −23 |
| | 0.2 | | 0.2 | | 0.1 | −27 |
| | 0.3 | | 0.2 | | 0.1 | −27 |
| | 0.5 | | 0.2 | | 0.1 | −27 |
| | 0.1 | | 0.3 | | 0.1 | −26 |
| | 0.2 | | 0.3 | | 0.1 | −27 |
| | 0.3 | | 0.3 | | 0.1 | −27 |
| | 0.5 | | 0.3 | | 0.1 | −21 |
| | 0.3 | LR10 | 0.35 | | 0.075 | −25 |
| | 0.3 | 2447/079 | 0.1 | | 0.075 | −28 |
| | 0.3 | 2447/084 | 0.2 | | 0.075 | −28 |
| | 0.3 | 2447/085 | 0.2 | | 0.075 | −28 |
| | 0.3 | 2447/086 | 0.2 | | 0.075 | −22 |
| | 0.3 | 2447/087 | 0.2 | | 0.075 | −26 |
| | 0.3 | 2447/088 | 0.2 | | 0.075 | −27 |
| | 0.3 | 2IN1 | 0.15 | Vamac 3038 | 0.025 | −21 |
| | 0.3 | | 0.15 | Vamac GLS | 0.025 | −22 |
| | 0.3 | | 0.15 | Vamac VCD | 0.025 | −26 |
| 2526/198 | 0.3 | | 0.15 | Vamac DP | 0.025 | −28 |
| | 0.3 | | 0.2 | | 0.075 | −25 |
| 2526/197 | 0.3 | | 0.2 | | 0.075 | −21 |
| 2680/064 | 0.3 | | 0.2 | | 0.075 | −31 |

CFI A 2526/211 is then tested with 2IN1 and Vamac DP in a biodiesel blend.

TABLE 19
CFI A Ery + CFI B + CFI C in B100 XME - Blends of biodiesel

| Biodiesel | | Dosage | | Dosage | | Dosage | CFPP |
|---|---|---|---|---|---|---|---|
| blend | CFI A Ery | w/v % | CFI B | w/v % | CFI C | w/v % | (° C.) |
| TME | None | — | None | — | None | — | 10 |
| | (control) | | (control) | | (control) | | |
| | 2526/211 | 0.3 | 2IN1 | 0.2 | Vamac DP | 0.075 | 10 |
| 70:30 | None | — | None | — | None | — | 2 |
| SME:TME | (control) | | (control) | | (control) | | |
| | 2526/211 | 0.3 | 2IN1 | 0.2 | Vamac DP | 0.075 | 0 |
| 60:40 | None | — | None | — | None | — | 4 |
| SME:TME | (control) | | (control) | | (control) | | |
| | 2526/211 | 0.3 | 2IN1 | 0.2 | Vamac DP | 0.075 | 0 |
| 50:50 | None | — | None | — | None | — | 5 |
| SME:TME | (control) | | (control) | | (control) | | |
| | 2526/211 | 0.3 | 2IN1 | 0.2 | Vamac DP | 0.075 | 4 |
| 75:25 | None | — | None | — | None | — | −10 |
| RME:SME | (control) | | (control) | | (control) | | |
| | 2526/211 | 0.3 | 2IN1 | 0.2 | Vamac DP | 0.075 | −19 |
| 80:20 | None | — | None | — | None | — | −9 |
| RME:SME | (control) | | (control) | | (control) | | |
| | 2526/211 | 0.3 | 2IN1 | 0.2 | Vamac DP | 0.075 | −14 |
| 95:5 | None | — | None | — | None | — | −9 |
| RME:SME | (control) | | (control) | | (control) | | |
| | 2526/211 | 0.3 | 2IN1 | 0.2 | Vamac DP | 0.075 | −12 |
| 90:10 | None | — | None | — | None | — | −10 |
| RME:PME | (control) | | (control) | | (control) | | |
| | 2526/211 | 0.3 | 2IN1 | 0.2 | Vamac DP | 0.075 | −15 |

Preferred esters of PFA+pentaerythritol derivatives in combination with preferred citric acid esters of monoglycerides and preferred copolymers of ethylene and an alkyl acrylate are dosed in biodiesel B7 RME and tested. The cold filter plugging point is measured and can be compared against the control diesel containing no additive.

TABLE 20
CFI A Ery + CFI B + CFI C in B7 RME The dosage
w/v % in the table is based on the biodiesel content
in the fuel (ie. in the total fuel CFI A Ery 0.021
w/v % and CFI B in a range between 0.01 and 0.024 w/v
% and CFI C between 0.002 and 0.005 w/v %)

| | Dosage | | Dosage | | Dosage | CFPP |
|---|---|---|---|---|---|---|
| CFI A Ery | w/v % | CFI B | w/v % | CFI C | w/v % | (° C.) |
| None | — | None | — | None | — | −29 |
| (control) | | (control) | | (control) | | |
| 2526/211 | 0.3 | 2IN1 | 0.2 | Vamac DP | 0.025 | −30 |
| | 0.3 | | 0.2 | | 0.075 | −34 |
| | 0.3 | | 0.15 | | 0.025 | −30 |
| | 0.3 | | 0.15 | | 0.075 | −31 |
| | 0.3 | | 0.15 | | 0.075 | −31 |
| | 0.3 | | 0.15 | | 0.025 | −31 |
| | 0.3 | | 0.15 | Vamac 3038 | 0.025 | −29 |
| | 0.3 | | 0.15 | Vamac GLS | 0.025 | −31 |
| | 0.3 | | 0.15 | Vamac VCD | 0.025 | −31 |
| | 0.3 | LR10 | 0.35 | Vamac DP | 0.075 | −31 |

CFI A 2526/211 is then tested with 2IN1 and Vamac DP in a further biodiesel blend.

TABLE 21
CFI A Ery + CFI B + CFI C in B10 XME blends of biodiesel The dosage
w/v % in the table is based on the biodiesel content in the fuel (ie. in
the total fuel CFI A Ery 0.03 w/v % and CFI B 0.02 w/v % and CFI C 0.0075 w/v %)

| Biodiesel | | Dosage | | Dosage | | Dosage | CFPP |
|---|---|---|---|---|---|---|---|
| blend | CFI A Ery | w/v % | CFI B | w/v % | CFI C | w/v % | (° C.) |
| 75:25 | None | — | None | — | None | — | −29 |
| RME:SME | (control) | | (control) | | (control) | | |
| | 2526/211 | 0.3 | 2IN1 | 0.2 | Vamac | 0.075 | −28 |
| | | | | | DP | | |
| 60:40 | None | — | None | — | None | — | −20 |
| SME:TME | (control) | | (control) | | (control) | | |
| | 2526/211 | 0.3 | 2IN1 | 0.2 | Vamac | 0.075 | −25 |
| | | | | | DP | | |
| 90:10 | None | — | None | — | None | — | −27 |
| RME:PME | (control) | | (control) | | (control) | | |
| | 2526/211 | 0.3 | 2IN1 | 0.2 | Vamac | 0.075 | −29 |
| | | | | | DP | | |

TABLE 22
B7 XME blends of biodiesel The dosage w/v % in the table is based
on the biodiesel content in the fuel (ie. in the total fuel CFI
A Ery 0.021 w/v % and CFI B 0.014 w/v % and CFI C 0.005 w/v %)

| Biodiesel | | Dosage | | Dosage | | Dosage | CFPP |
|---|---|---|---|---|---|---|---|
| blend | CFI A Ery | w/v % | CFI B | w/v % | CFI C | w/v % | (° C.) |
| 75:25 | None | — | None | — | None | — | −29 |
| RME:SME | (control) | | (control) | | (control) | | |
| | 2526/211 | 0.3 | 2IN1 | 0.2 | Vamac | 0.075 | −28 |
| | | | | | DP | | |
| 60:40 | None | — | None | — | None | — | −20 |
| SME:TME | (control) | | (control) | | (control) | | |
| | 2526/211 | 0.3 | 2IN1 | 0.2 | Vamac | 0.075 | −28 |
| | | | | | DP | | |
| 90:10 | None | — | None | — | None | — | −27 |
| RME:PME | (control) | | (control) | | (control) | | |
| | 2526/211 | 0.3 | 2IN1 | 0.2 | Vamac | 0.075 | −31 |
| | | | | | DP | | |

B10 XME is a biodiesel containing 10% biodiesel and 90% petro diesel.

### Application in Biofuels CFI A Hex

A range of esters of PFA+hexaglycerol dosed in biodiesel B100 RME are tested. The cold filter plugging point is measured and can be compared against the control diesel containing no additive.

TABLE 23
CFI A Hex in B100 RME

| CFI A Hex | Dosage w/v % | CFPP (° C.) |
|---|---|---|
| None (control) | — | −14 |
| 2526/173 | 0.2 | −17 |
| | 0.5 | −20 |
| | 1.0 | −20 |
| | 2.0 | −17 |
| 2680/025 | 0.2 | −15 |
| | 0.5 | −20 |
| | 1.0 | −17 |
| | 2.0 | −13 |
| 2526/209 | 0.075 | −16 |
| | 0.5 | −17 |
| | 0.75 | −17 |
| | 1.0 | −20 |
| 2526/186 | 0.2 | −16 |
| | 0.5 | −16 |
| | 1.0 | −15 |
| | 2.0 | −16 |
| 2526/150 | 0.075 | −21 |
| | 0.2 | −17 |
| | 0.5 | −17 |
| | 1.0 | −20 |
| | 2.0 | −17 |
| 2526/147 | 0.2 | −16 |
| | 0.5 | −16 |
| | 1.0 | −18 |
| | 2.0 | −18 |
| 2526/172 | 0.2 | −17 |
| | 0.5 | −17 |
| | 1.0 | −19 |
| | 2.0 | −19 |
| 2349/140 | 0.2 | −14 |
| | 0.5 | −13 |
| | 1.0 | −13 |
| | 2.0 | −14 |
| 2349/141 | 0.2 | −15 |
| | 0.5 | −14 |
| | 1.0 | −14 |
| | 2.0 | −15 |
| 2863/017 | 0.2 | −11 |
| | 0.5 | −13 |
| | 1.0 | −14 |
| | 2.0 | −14 |

A preferred ester of PFA+hexaglycerol is then dosed in biodiesel B100 TME and tested. The cold filter plugging point is measured and can be compared against the control diesel containing no additive. As discussed above, B100 TME is a 100% biodiesel.

TABLE 24
B100 TME

| CFS A Hex | Dosage w/v % | CFPP (° C.) |
|---|---|---|
| None (control) | — | 10 |
| 2526/209 | 0.2 | 11 |
| | 0.5 | 10 |
| | 0.75 | 10 |
| | 1.0 | 10 |
| | 2.0 | 10 |

### Application in Biofuels CFI A Hex+CFI B

Combinations of (i) ester of PFA+hexaglycerol and (ii) Citrems are then dosed in bio diesel blends and tested. The cold filter plugging point is measured and can be compared against the control diesel containing no additive. As discussed above B100RME is a 100% biodiesel.

TABLE 25
CFI A Hex + CFI B in B100 RME

| | Dosage | | Dosage | |
|---|---|---|---|---|
| CFI A Hex | w/v % | CFI B | w/v % | CFPP (° C.) |
| None (control) | — | None (control)- | — | −14 |
| 2680/025 | 0.3 | 2IN1 | 0.2 | −20 |
| | 0.3 | | 0.5 | −20 |
| 2526/150 | 1.0 | | 1.0 | −16 |
| | 2.0 | | 2.0 | −16 |

### Application in Biofuels CFI A Hex+CFI C

A preferred ester of PFA+hexaglycerol in combination with preferred copolymers of ethylene and an alkyl acrylate are dosed in bio diesel B100 RME and tested. The cold filter plugging point is measured and can be compared against the control diesel containing no additive.

TABLE 26
B100 RME - Preferred biodiesel

| | Dosage | | Dosage | |
|---|---|---|---|---|
| CFI A Hex | w/v % | CFI C | w/v % | CFPP (° C.) |
| None (control) | — | None (control) | — | −14 |
| 2526/173 | 1.0 | Vamac DP | 0.2 | −16 |
| | 0.5 | | 1.0 | −12 |
| | 2.0 | | 1.0 | −10 |
| 2526/209 | 0.5 | | 0.075 | −20 |
| | 0.5 | | 0.01 | −19 |
| | 0.5 | | 0.02 | −20 |
| | 0.5 | | 0.03 | −20 |
| | 0.5 | | 0.04 | −20 |
| | 0.5 | | 0.05 | −20 |
| | 0.5 | | 0.06 | −21 |
| | 1.0 | | 0.075 | −19 |
| | 1.0 | | 0.05 | −20 |

A preferred ester of PFA+hexaglycerol in combination with preferred copolymers of ethylene and an alkyl acrylate are dosed in biodiesel B100 XME and tested. The cold filter plugging point is measured and can be compared against the control diesel containing no additive.

TABLE 27
B100 XME - Blends of different biodiesel

| | Dosage | | Dosage | |
|---|---|---|---|---|
| CFI A Hex | w/v % | CFI C | w/v % | CFPP (° C.) |
| None (control) | — | None (control) | — | 10 |
| 2526/209 | 0.075 | Vamac DP | 0.05 | 10 |
| | 0.075 | | 0.075 | 10 |
| | 1.0 | | 0.05 | 10 |
| | 1.0 | | 0.075 | 10 |

### Application in Biofuels CFI A Hex+CFI B+CFI C

Preferred esters of PFA+pentaerythritol derivatives in combination with preferred citric acid esters of monoglycerides and preferred copolymers of ethylene and an alkyl acrylate are dosed in biodiesel B7 RME and tested. The cold filter plugging point is measured and can be compared against the control diesel containing no additive.

TABLE 28
CFI A Hex + CFI B + CFI C in B100 RME

| | Dosage | | Dosage | | Dosage | CFPP |
|---|---|---|---|---|---|---|
| CFI A Hex | w/v % | CFI B | w/v % | CFI C | w/v % | (° C.) |
| None | — | None | — | None | — | −14 |
| (control) | | (control) | | (control) | | |
| 2680/025 | 0.5 | 2IN1 | 0.2 | Vamac DP | 0.075 | −21 |
| 2526/209 | 0.8 | | 0.2 | | 0.075 | −20 |

### Application in Biofuels CFI A

A range of esters of PFA+other polyols dosed in bio diesel B100 RME are tested. The cold filter plugging point is measured and can be compared against the control diesel containing no additive.

TABLE 29
CFI A Poly in B100 RME

| CFI A Poly | Dosage w/v % | CFPP (° C.) |
|---|---|---|
| 2461/187 | 0.5 | −17 |
| | 1.0 | −18 |
| 2526/191 | 0.2 | −17 |
| | 0.5 | −18 |
| | 1.0 | −18 |
| | 2.0 | −18 |
| 2526/192 | 0.2 | −15 |
| | 0.5 | −16 |
| | 1.0 | −17 |
| | 2.0 | −16 |
| 2526/205 | 0.2 | −15 |
| | 0.5 | −15 |
| | 1.0 | −17 |
| | 2.0 | −17 |
| 2526/194 | 0.2 | −14 |
| | 0.5 | −15 |
| | 1.0 | −14 |
| | 2.0 | −14 |
| 2525/204 | 0.2 | −16 |
| | 0.5 | −16 |
| | 1.0 | −16 |
| | 2.0 | −19 |
| 2526/185 | 0.2 | −15 |
| | 0.5 | −17 |
| | 1.0 | −15 |
| | 2.0 | −12 |
| 2526/187 | 0.2 | −16 |
| | 0.5 | −18 |
| | 1.0 | −18 |
| | 2.0 | −16 |

### Application in Biofuels CFI B

Preferred citric acid esters of monoglycerides are dosed in biodiesel B100 RME and tested. The cold filter plugging point is measured and can be compared against the control diesel containing no additive.

TABLE 30

| CFI B | Dosage (w/v %) | CFPP (° C.) |
|---|---|---|
| Citrem 2IN1 | 2.0 | −19 |
| Citrem 2IN1 | 0.2 | −11 |
| Citrem LR10 extra | 3.0 | −16 |
| Citrem LR10 extra | 0.2 | −11 |

### Application in Biofuels CFI C

Preferred copolymers of ethylene and an alkyl acrylate are dosed in biodiesel B100 RME and tested. The cold filter plugging point is measured and can be compared against the control diesel containing no additive.

TABLE 31

| CFI C | Dosage (w/v %) | CFPP (° C.) |
|---|---|---|
| Elvaloy EAC 34035 | 0.075 | −15 |
| Vamac DP | 0.075 | −16 |

### Application in Biofuels CFI B+CFI C

Preferred citric acid esters of monoglycerides and preferred copolymers of ethylene and an alkyl acrylate are dosed in biodiesel B100 RME and tested. The cold filter plugging point is measured and can be compared against the control diesel containing no additive.

TABLE 32

| | Dosage | | Dosage | |
|---|---|---|---|---|
| CFI B | (w/v %) | CFI C | (w/v %) | CFPP (° C.) |
| 2447/086 | 0.2 | Vamac DP | 0.075 | −22 |

### CONCLUSIONS

We have shown that an ester of a polyol and a fatty acid oligomer is effective in reducing the cold filter plugging point of fuels, such as diesel and in particular biodiesels.

When a combined product is provided which further includes a citric acid ester of a monoglyceride we have effectively decreased the cold filter plugging point (CFPP) in biodiesel rapeseed methyl esters (RME) to −30° C.

It is believed that the ester of a polyol and a fatty acid oligomer acts as an anticrystallizer and the citric acid ester of a monoglyceride is an emulsifier which has a dual action impacting the anticrystallization and crystal growth.

Furthermore when a further component, namely a copolymer of ethylene and an alkyl acrylate, is added further effects are seen. These combination additives can reduce the CFPP from −29° C. to −34° C. in B7 based on 7% RME (7% biodiesel in petro diesel).

The invention will be described in further detail in the following numbered paragraphs. The present invention provides:
1. A compound which is an ester of

(i) a polyol wherein the polyol is selected from at least pentaerythritol, polymers thereof and mixtures thereof; and

(ii) a fatty acid oligomer, wherein the fatty acid oligomer has a degree of polymerisation of from 2 to 7.
2. A compound according to paragraph 1 wherein the polyol is at least a polymer of pentaerythritol.
3. A compound according to paragraph 2 wherein the polymer of pentaerythritol has a degree of polymerisation of from greater than 1 to no greater than 10.
4. A compound according to paragraph 3 wherein the polymer of pentaerythritol has a degree of polymerisation of from 2 to 10.
5. A compound according to paragraph 3 wherein the polymer of pentaerythritol has a degree of polymerisation of from 2 to 5.
6. A compound according to any one of the preceding paragraphs wherein the polyol has a hydroxyl value of from 850 to 1830, preferably from 950 to 1300.
7. A compound according to any one of the preceding paragraphs wherein the polyol has a longest chain length of carbons and oxygen of from 7 to 15 atoms.
8. A compound according to any one of the preceding paragraphs wherein the polyol has from 3 to 12 hydroxyl groups, preferably from 3 to 10 hydroxyl groups.
9. A compound according to any one of the preceding paragraphs wherein the polyol comprises at least polypentaerythritol.
10. A compound according to any one of the preceding paragraphs wherein the polyol comprises at least one polyol selected from pentaerythritol, dipentaerythritol, tripentaerythritol, and combinations thereof.
11. A compound according to any one of the preceding paragraphs wherein the polyol is at least dipentaerythritol.
12. A compound according to any one of the preceding paragraphs wherein the polyol further comprises a polyol selected from glycerol, polymers thereof and mixtures thereof.
13. A compound according to any one of the preceding paragraphs wherein the polyol further comprises glycerol.
14. A compound according to any one of the preceding paragraphs wherein the polyol is at least a mixture of glycerol and pentaerythritol or a polymer thereof.
15. A compound according to any one of the preceding paragraphs wherein the polyol is at least a mixture of dipentaerythritol and glycerol.
16. A compound according to any one of the preceding paragraphs wherein the polyol is at least a compound of Formula I

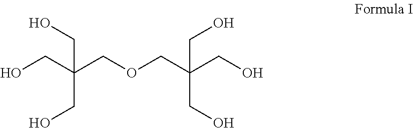 [SMILES: OCC(CO)(CO)COCC(CO)(CO)CO]

17. A compound according to paragraph 16 wherein the polyol is at least a compound of Formula I in an amount of at least 50 wt % based on the amount of polyols.
18. A compound according to paragraph 16 wherein the polyol is at least a compound of Formula I in an amount of at least 70 wt % based on the amount of polyols.
19. A compound according to paragraph 16 wherein the polyol is at least a compound of Formula I in an amount of at least 80 wt % based on the amount of polyols.
20. A compound according to any one of the preceding paragraphs wherein the fatty acid oligomer has a degree of polymerisation of from 2 to 5.
21. A compound according to any one of the preceding paragraphs wherein the fatty acid oligomer is prepared from at least one fatty acid having from 6 to 30 carbon atoms.
22. A compound according to any one of the preceding paragraphs wherein the fatty acid oligomer is prepared from at least one fatty acid having a hydroxyl group on the carbon chain of the fatty acid.
23. A compound according to any one of the preceding paragraphs wherein the fatty acid oligomer is prepared from a mixture of at least

(i) a fatty acid having a hydroxyl group on the carbon chain of the fatty acid and

(ii) an analogous fatty acid without said hydroxyl substitution.
24. A compound according to any one of the preceding paragraphs wherein the fatty acid oligomer is prepared from a mixture of at least

(i) a C18-OH fatty acid having a hydroxyl group on the carbon chain of the fatty acid and

(ii) a C18 fatty acid without said hydroxyl substitution.
25. A compound according to any one of the preceding paragraphs wherein the fatty acid oligomer is prepared from at least an unsaturated fatty acid having a hydroxyl group on the carbon chain of the fatty acid.
26. A compound according to any one of the preceding paragraphs wherein the fatty acid oligomer is prepared from at least an unsaturated fatty acid having a hydroxyl group on the carbon chain of the fatty acid,

wherein the unsaturated fatty acid having a hydroxyl group on the carbon chain of the fatty acid is present in an amount of no greater than 50 wt. % based on the total weight of fatty acids used to prepare the fatty acid oligomer.
27. A compound according to any one of the preceding paragraphs wherein the fatty acid oligomer is prepared from a mixture of at least

(i) a saturated fatty acid having a hydroxyl group on the carbon chain of the fatty acid,

(ii) an unsaturated fatty acid having a hydroxyl group on the carbon chain of the fatty acid.
28. A compound according to any one of the preceding paragraphs wherein the fatty acid oligomer is prepared from at least 12-hydroxy stearic acid.
29. A compound according to any one of the preceding paragraphs wherein the fatty acid oligomer is prepared from at least ricinoleic acid.
30. A compound according to any one of the preceding paragraphs wherein the fatty acid oligomer is prepared from a mixture of at least

(i) 12-hydroxy stearic acid and

(ii) ricinoleic acid.
31. A compound according to any one of the preceding paragraphs wherein the fatty acid oligomer is prepared from a mixture of at least

(i) 12-hydroxy stearic acid in an amount of 60-90 wt % based on the total weight of fatty acids used to prepare the fatty acid oligomer and

(ii) ricinoleic acid in an amount of 10-40 wt % based on the total weight of fatty acids used to prepare the fatty acid oligomer.
32. A compound according to any one of the preceding paragraphs wherein the fatty acid oligomer is prepared from a mixture of at least

(i) 12-hydroxy stearic acid in an amount of 70-90 wt % based on the total weight of fatty acids used to prepare the fatty acid oligomer and

(ii) ricinoleic acid in an amount of 10-30 wt % based on the total weight of fatty acids used to prepare the fatty acid oligomer.
33. A compound according to any one of the preceding paragraphs wherein the fatty acid oligomer is prepared from a mixture of at least

(i) 12-hydroxy stearic acid in an amount of 75-85 wt % based on the total weight of fatty acids used to prepare the fatty acid oligomer and

(ii) ricinoleic acid in an amount of 15-25 wt % based on the total weight of fatty acids used to prepare the fatty acid oligomer.
34. A compound according to any one of the preceding paragraphs wherein the fatty acid oligomer is prepared from a mixture of at least

(i) 12-hydroxy stearic acid in an amount of approximately 80 wt % based on the total weight of fatty acids used to prepare the fatty acid oligomer and

(ii) ricinoleic acid in an amount of approximately 20 wt % based on the total weight of fatty acids used to prepare the fatty acid oligomer.
35. A compound according to any one of the preceding paragraphs wherein the fatty acid oligomer is prepared from a mixture which further comprises a fatty acid group which does not contain a hydroxyl group on the fatty acid chain.
36. A compound according to any one of the preceding paragraphs wherein the fatty acid oligomer is prepared from a mixture of at least

(i) an unsaturated fatty acid having a hydroxyl group on the carbon chain of the fatty acid and

(ii) an analogous unsaturated fatty acid without said hydroxyl substitution.
37. A compound according to any one of the preceding paragraphs wherein the fatty acid oligomer is prepared from a mixture of at least

(i) a saturated fatty acid having a hydroxyl group on the carbon chain of the fatty acid and

(ii) an analogous saturated fatty acid without said hydroxyl substitution.
38. A compound according to any one of the preceding paragraphs wherein the fatty acid oligomer is prepared from a mixture of at least

(i) an unsaturated fatty acid having a hydroxyl group on the carbon chain of the fatty acid;

(ii) an unsaturated fatty acid analogous to (i) without said hydroxyl substitution;

(iii) a saturated fatty acid having a hydroxyl group on the carbon chain of the fatty acid; and

(iv) a saturated fatty acid analogous to (iii) without said hydroxyl substitution.
39. A compound according to any one of the preceding paragraphs wherein the fatty acid oligomer has a degree of polymerisation of from 2 to 5 when measured by NMR.
40. A compound according to any one of the preceding paragraphs wherein the fatty acid oligomer has an acid value of 20 to 100.
41. A compound according to any one of the preceding paragraphs wherein the fatty acid oligomer has an acid value of 40 to 80.
42. A compound according to any one of the preceding paragraphs wherein the ratio of polyol to fatty acid oligomer based on weight is from 1:50 to 1:1
43. A compound according to any one of the preceding paragraphs wherein the ratio of polyol to fatty acid oligomer based on weight is from 1:25 to 1:4.
44. A compound according to any one of the preceding paragraphs wherein the compound is of Formula II

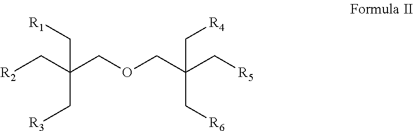

wherein each of R₁ to R₆ is independently selected from —OH and fatty acid oligomer esters, wherein at least one of R₁ to R₆ is a fatty acid oligomer ester.
45. A compound according to paragraph 44 wherein each of R₁ to R₆ is independently selected from —OH and fatty acid oligomers of Formula III

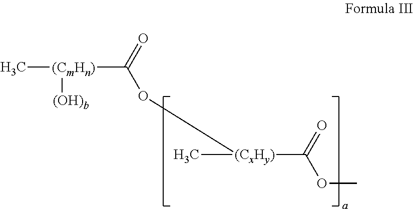

wherein b is 0 or 1, m is an integer from 0 to 28, n is selected from 2m-b, 2m-2-b, 2m-4-b, x is an integer from 0 to 28, y is selected from 2x-1, 2x-3, 2x-5, and a is an integer from 1 to 9.
46. A composition comprising

(a) a compound as defined in any one of paragraphs 1 to 45

and

(b) a citric acid ester of a monoglyceride

or

(c) a copolymer of ethylene and an alkyl acrylate
47. A composition according to paragraph 46 comprising

(a) a compound as defined in any one of paragraphs 1 to 45 and

(b) a citric acid ester of a monoglyceride.
48. A composition according to paragraph 47 wherein the citric acid ester of a monoglyceride is a citric acid ester of a monoglyceride derived from an oil selected from sunflower oil, high oleic sunflower oil or rapeseed oil.
49. A composition according to paragraph 47 or 48 wherein the ratio of (a) to (b) based on weight is from 20:1 to 1:10.
50. A composition according to paragraph 49 wherein the ratio of (a) to (b) based on weight is from 10:1 to 1:3.
51. A composition according to paragraph 46 comprising

(a) a compound as defined in any one of paragraphs 1 to 45 and

(c) a copolymer of ethylene and an alkyl acrylate.
52. A composition according to paragraph 51 wherein the alkyl acrylate has up to 10 carbon atoms in the alkyl chain.
53. A composition according to paragraph 51 or 52 wherein the alkyl group of the alkyl acrylate is selected from methyl, ethyl, n-butyl and 2-ethylhexyl.
54. A composition according to paragraph 51, 52 or 53 wherein the alkyl acrylate is selected from the group consisting of methyl acrylate, ethyl acrylate, n-butyl acrylate, 2-ethylhexyl acrylate and mixtures thereof.
55. A composition according to any one of paragraphs 51 to 54 wherein the alkyl acrylate is methyl acrylate.
56. A composition according to any one of paragraphs 51 to 55 wherein the ratio of (a) to (c) based on weight is from 100:1 to 1:2.
57. A composition according to paragraph 56 wherein the ratio of (a) to (c) based on weight is from 50:1 to 1:1.
58. A composition according to any one of paragraphs 46 to 55 comprising

(a) a compound as defined in any one of paragraphs 1 to 45;

(b) a citric acid ester of a monoglyceride; and

(c) a copolymer of ethylene and an alkyl acrylate.
59. A composition according to paragraph 58 wherein

the ratio of (a) to (b) based on weight is from 20:1 to 1:10; and

the ratio of (a) to (c) based on weight is from 100:1 to 1:2.
60. A composition according to paragraph 58 wherein

the ratio of (a) to (b) based on weight is from 10:1 to 1:3; and

the ratio of (a) to (c) based on weight is from 50:1 to 1:1.
61. A cold flow improver comprising a compound as defined in any one of paragraphs 1 to 45 or a composition as defined in any one of paragraphs 46 to 60.
62. A fuel composition comprising:

(a) a fuel;

(b) a compound which is an ester of

(I) a polyol wherein the polyol has at least three hydroxyl groups; and

(II) a fatty acid oligomer, wherein the fatty acid oligomer has a degree of polymerisation from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid.
63. A fuel composition according to paragraph 62 wherein the polyol is a polymer of an alcohol.
64. A fuel composition according to paragraph 62 or 63 wherein the polymer of the alcohol has a degree of polymerisation of from greater than 1 to no greater than 10.
65. A fuel composition according to paragraph 62, 63 or 64 wherein the polyol is a polymer of at least dipentaerythritol.
66. A fuel composition according to any one of paragraphs 62 to 66 wherein the polyol is a polymer of at least glycerol.
67. A fuel composition according to any one of paragraphs 62 to 66 wherein the polyol is a polymer of at least glycerol and dipentaerythritol.
68. A fuel composition according to any of paragraphs 62 to 67 wherein the polyol is branched polyol.
69. A fuel composition according to any of paragraphs 62 to 68 wherein the polyol has a hydroxyl value of from 850 to 1830, preferably from 950 to 1300.
70. A fuel composition according to any of paragraphs 62 to 69 wherein the polyol has a longest chain length of carbons and oxygen of from 7 to 30 atoms.
71. A fuel composition according to any of paragraphs 62 to 70 wherein the polyol has from 3 to 12 hydroxyl groups, preferably from 3 to 10 hydroxyl groups.
72. A fuel composition according to any of paragraphs 62 to 71 comprising

(a) a fuel;

(b) a compound as defined in any one of paragraphs 1 to 45 or a composition as defined in any one of paragraphs 46 to 60.
73. A fuel composition according to any of paragraphs 62 to 72 wherein the fuel is selected from diesels, heavy fuel oils, marine gasoils and kerosene.
74. A fuel composition according to paragraph 73 wherein the fuel is a diesel.
75. A fuel composition according to paragraph 74 wherein the diesel is biodiesel or a biodiesel blend.
76. A process for reducing, preventing or inhibiting cold filter plugging in a diesel engine, comprising the step of: dosing a fuel with a compound which is an ester of

(I) a polyol wherein the polyol has at least three hydroxyl groups; and

(II) a fatty acid oligomer, wherein the fatty acid oligomer has a degree of polymerisation from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid.
77. A process according to paragraph 76 wherein the polyol is a polymer of an alcohol.
78. A process according to paragraph 76 or 77 wherein the polymer of the alcohol has a degree of polymerisation from greater than 1 to no greater than 10.
79. A process according to paragraph 76, 77 or 78 wherein the polyol is a polymer of at least pentaerythritol.
80. A process according to any one of paragraphs 76 to 79 wherein the polyol is a polymer of at least glycerol.
81. A process according to any one of paragraphs 76 to 80 wherein the polyol is a polymer of at least glycerol and dipentaerythritol.
82. A process according to any one of paragraphs 76 to 81 wherein the polyol is branched polyol.
83. A process according to any one of paragraphs 76 to 82 wherein the polyol has a hydroxyl value of from 850 to 1830, preferably from 950 to 1300.
84. A process according to any one of paragraphs 76 to 83 wherein the polyol has a longest chain length of carbons and oxygen of from 7 to 30 atoms.
85. A process according to any one of paragraphs 76 to 84 wherein the polyol has from 3 to 12 hydroxyl groups, preferably from 3 to 10 hydroxyl groups.
86. A process according to any one of paragraphs 76 to 85 wherein the fuel is dosed with a compound as defined in any one of paragraphs 1 to 45 or a composition as defined in any one of paragraphs 46 to 60.
87. Use of a compound for reducing, preventing or inhibiting cold filter plugging in a diesel engine wherein the compound is an ester of

(I) a polyol wherein the polyol has at least three hydroxyl groups; and

(II) a fatty acid oligomer, wherein the fatty acid oligomer has a degree of polymerisation from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid.
88. A use according to paragraph 87 wherein the polyol is a polymer of an alcohol.
89. A use according to paragraph 87 or 88 wherein the polymer of the alcohol has a degree of polymerisation from greater than 1 to no greater than 10.
90. A use according to paragraph 87, 88 or 89 wherein the polyol is a polymer of at least dipentaerythritol.
91. A use according to any one of paragraphs 87 to 90 wherein the polyol is a polymer of at least glycerol.
92. A use according to any one of paragraphs 87 to 91 wherein the polyol is a polymer of at least glycerol and dipentaerythritol.
93. A use according to any of paragraphs 87 to 92 wherein the polyol is branched polyol.
94. A use according to any of paragraphs 87 to 93 wherein the polyol has a hydroxyl value of from 850 to 1830, preferably from 950 to 1300.
95. A use according to any of paragraphs 87 to 94 wherein the polyol has a longest chain length of carbons and oxygen of from 7 to 30 atoms.
96. A use according to any of paragraphs 87 to 95 wherein the polyol has from 3 to 12 hydroxyl groups, preferably from 3 to 10 hydroxyl groups.
97. A use according to any of paragraphs 87 to 95 wherein a compound is as defined in any one of paragraphs 1 to 45 or is in a composition as defined in any one of paragraphs 46 to 60.
98. A compound substantially as hereinbefore described with reference to any one of the Examples.
99. A composition substantially as hereinbefore described with reference to any one of the Examples.
100. A fuel composition substantially as hereinbefore described with reference to any one of the Examples.
101. A process substantially as hereinbefore described with reference to any one of the Examples.
102. A use substantially as hereinbefore described with reference to any one of the Examples.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims

1. A compound which is an ester of
(i) a polyol wherein the polyol is selected from at least pentaerythritol, polymers thereof and mixtures thereof; and
(ii) a fatty acid oligomer, wherein the fatty acid oligomer has a degree of polymerisation of from 2 to 7.

2. A compound according to claim 1 wherein the polyol comprises at least one polyol selected from pentaerythritol, dipentaerythritol, tripentaerythritol, and combinations thereof.

3. A compound according to claim 1 wherein the polyol further comprises glycerol.

4. A compound according to claim 1 wherein the polyol is at least a mixture of dipentaerythritol and glycerol.

5. A compound according to claim 1 wherein the polyol is at least a compound of Formula I

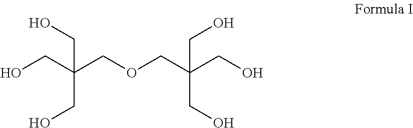 [SMILES: OCC(CO)(CO)COCC(CO)(CO)CO]

6. A compound according to claim 5 wherein the polyol is at least a compound of Formula I in an amount of at least 50 wt % based on the amount of polyols.

7. A compound according to claim 1 wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid.

8. A compound according to claim 1 wherein the fatty acid oligomer is prepared from at least 12-hydroxy stearic acid.

9. A compound according to claim 1 wherein the fatty acid oligomer is prepared from at least ricinoleic acid.

10. A compound according to claim 1 wherein the fatty acid oligomer is prepared from a mixture of at least
(i) 12-hydroxy stearic acid and
(ii) ricinoleic acid.

11. A compound according to claim 1 wherein the fatty acid oligomer is prepared from a mixture of at least
(i) 12-hydroxy stearic acid in an amount of 60-90 wt % based on the total weight of fatty acids used to prepare the fatty acid oligomer and
(ii) ricinoleic acid in an amount of 10-40 wt % based on the total weight of fatty acids used to prepare the fatty acid oligomer.

12. A compound according to claim 1 wherein the fatty acid oligomer is prepared from a mixture which further comprises a fatty acid group which does not contain a hydroxyl group on the fatty acid chain.

13. A compound according to claim 1 wherein the fatty acid oligomer has a degree of polymerisation of from 2 to 5 when measured by NMR.

14. A composition comprising
(a) a compound as defined in claim 1
and
(b) a citric acid ester of a monoglyceride
or
(c) a copolymer of ethylene and an alkyl acrylate.

15. A composition according to claim 14 comprising
(a) said compound and
(b) a citric acid ester of a monoglyceride.

16. A composition according to claim 15 comprising
(a) said compound and
(c) a copolymer of ethylene and an alkyl acrylate.

17. A composition according to claim 14 wherein the alkyl acrylate is methyl acrylate.

18. A cold flow improver comprising a compound as defined in claim 1.

19. A fuel composition comprising:
(a) a fuel;
(b) a compound which is an ester of
(I) a polyol wherein the polyol has at least three hydroxyl groups; and
(II) a fatty acid oligomer, wherein the fatty acid oligomer has a degree of polymerisation from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid.

20. A fuel composition according to claim 19 comprising
(a) a fuel;
(b) a compound which is an ester of
(i) a polyol wherein the polyol is selected from at least pentaerythritol, polymers thereof and mixtures thereof; and
(ii) a fatty acid oligomer, wherein the fatty acid oligomer has a degree of polymerisation of from 2 to 7.

21. A fuel composition according to claim 20 wherein the diesel is biodiesel or a biodiesel blend.

22. A process for reducing, preventing or inhibiting cold filter plugging in a diesel engine, comprising the step of: dosing a fuel with a compound which is an ester of
(I) a polyol wherein the polyol has at least three hydroxyl groups; and
(II) a fatty acid oligomer, wherein the fatty acid oligomer has a degree of polymerisation from 2 to 7, and wherein the fatty acid oligomer is prepared from a mixture of at least
(i) a saturated fatty acid having a hydroxyl group on the carbon chain of the fatty acid,
(ii) an unsaturated fatty acid having a hydroxyl group on the carbon chain of the fatty acid.

23. A process according to claim 22 wherein the polyol is a polymer of at least glycerol and dipentaerythritol.

24-25. (canceled)
